(19) United States
(12) Patent Application Publication
(10) Pub. No.: US 2026/0036540 A1
(43) Pub. Date: Feb. 5, 2026

(54) SENSOR WITH ENHANCED DURABILITY USING GRAPHENE INK FORMULATION

(71) Applicant: Lyten, Inc., San Jose, CA (US)

(72) Inventors: Paul Biggins, San Jose, CA (US); Jacques Nicole, Palo Alto, CA (US); Brian Ishaug, Pleasanton, CA (US); Jaewan Mun, Mountain View, CA (US)

(21) Appl. No.: 19/286,102

(22) Filed: Jul. 30, 2025

Related U.S. Application Data
(60) Provisional application No. 63/678,933, filed on Aug. 2, 2024.

(51) Int. Cl. G01N 27/12; G01N 33/00; G01N 33/48

(52) U.S. Cl. CPC G01N 27/125; G01N 33/0027; G01N 33/48

(57) ABSTRACT

The disclosed sensor comprises multiple types of graphene ink. The graphene ink may be applied via individual layers, where each layer is of a different type of graphene. Additionally, the graphene ink may be applied via a layer where the graphene ink is a mixture of two or more types of graphene. In either scenario, conductive patches of graphene and low conductivity interstitial carbon material may be created in the resulting material of the sensor. The low conductivity interstitial carbon material bridges the large, conductive patches of graphene, providing a connection between them. The graphene ink includes a first type of graphene configured for conductivity and a second type of graphene configured for wear and tear resistance. The sensor can be a resonant sensor, a vapor or gas sensor, a biosensor, or a printed label sensor, among others.

### RELATED APPLICATIONS

The present application is related, and claims priority to, U.S. Provisional Patent Application No. 63/678,933, filed Aug. 2, 2024 and entitled “SENSOR WITH ENHANCED DURABILITY USING GRAPHENE INK FORMULATION”, the contents of which are herein incorporated by reference in entirety.

### FIELD OF THE INVENTION

The present disclosure relates to sensor technology, and more particularly to a sensor comprising multiple layers of graphene ink configured for enhanced durability and electrical conductivity.

### BACKGROUND

Currently, graphene is a popular material for sensor construction due to its high surface area, excellent electrical conductivity, and mechanical flexibility. In particular, graphene can be applied onto a substrate in the form of an ink. Conductive inks are used in printed electronics to create conductive traces, a core component of many electronic devices. The properties of the graphene ink, such as its conductivity and flexibility, can be influenced by the formulation of the ink, including the types and proportions of the components used.

Despite the promising properties of graphene and its potential in sensor applications, challenges remain. One such challenge is the susceptibility of graphene to cracking, which can lead to electrical discontinuities and impair the functionality, durability, and performance of the sensor.

As such, there is thus a need for addressing these and/or other issues associated with the prior art.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a sensor includes a plurality of layers of graphene ink. Each layer of graphene ink comprises a mixture of large, conductive patches of graphene and low conductivity interstitial carbon material. The low conductivity interstitial carbon material bridges the large, conductive patches of graphene.

According to other aspects of the present disclosure, the sensor may include one or more of the following features. The graphene ink may comprise a first type of graphene configured for conductivity and a second type of graphene configured for wear and tear resistance. The first type of graphene and the second type of graphene may be mixed together prior to application on the sensor. Alternatively, the first type of graphene and the second type of graphene may be applied in separate layers on the sensor. The first type of graphene may be applied before the second type of graphene, or the second type of graphene may be applied before the first type of graphene. The low conductivity interstitial carbon material may comprise a carbon-based filler material. The large, conductive patches of graphene and the low conductivity interstitial carbon material may be arranged in a non-overlapping pattern.

According to another aspect of the present disclosure, the sensor may be a resonant sensor. According to other aspects of the present disclosure, the sensor may be a vapor or gas sensor, a biosensor, or a printed label sensor. The sensor may be configured to detect changes in environmental, physical, chemical, biological, electrical, thermal, mechanical, or optical conditions.

In some aspects, the techniques described herein relate to a sensor, wherein the sensor is configured to detect changes in at least one of: environmental conditions, physical conditions, chemical conditions, biological conditions, electrical conditions, thermal conditions, mechanical conditions, or optical conditions.

In some aspects, the techniques described herein relate to a sensor, including: a graphene ink formulation, wherein the graphene ink formulation includes: a first type of graphene configured for conductivity and a second type of graphene configured for wear and tear resistance, wherein the first type of graphene and the second type of graphene are mixed together prior to application on the sensor.

In some aspects, the techniques described herein relate to a sensor, wherein the first type of graphene is configured for high conductivity and the second type of graphene is configured for high elasticity.

In some aspects, the techniques described herein relate to a sensor, wherein the first type of graphene and the second type of graphene are mixed together prior to application on the sensor.

In some aspects, the techniques described herein relate to a sensor, wherein the first type of graphene and the second type of graphene are applied in separate layers on the sensor.

In some aspects, the techniques described herein relate to a sensor, including: a graphene ink formulation, wherein the graphene ink formulation includes: a first type of graphene configured for conductivity and a second type of graphene configured for wear and tear resistance, wherein the first type of graphene and the second type of graphene are applied in separate layers on the sensor.

In some aspects, the techniques described herein relate to a sensor, wherein the first type of graphene is configured for high conductivity and the second type of graphene is configured for high elasticity.

In some aspects, the techniques described herein relate to a sensor, wherein the first type of graphene and the second type of graphene are mixed together prior to application on the sensor.

In some aspects, the techniques described herein relate to a sensor, wherein each of the first type of graphene and the second type of graphene are mixtures each containing two or more types of graphene.

The foregoing general description of the illustrative embodiments and the following detailed description thereof are merely exemplary aspects of the teachings of this disclosure and are not restrictive.

### BRIEF DESCRIPTION OF THE DRAWINGS

FIG. 1 illustrates a process flowchart for creating a conductive graphene ink with low degradation, according to aspects of the present disclosure.

FIG. 2 depicts an orthogonal view of a graphene formation showing distinct configurations, according to aspects of the present disclosure.

FIG. 3 presents an isometric view of a graphene based material, according to aspects of the present disclosure.

FIG. 4 shows an S-parameters graph demonstrating the relationship between island size and S-parameter magnitude, according to aspects of the present disclosure.

FIG. 5 depicts a graph of S-parameters comparing the performance of a self-healing sensor and a discontinuous sensor, according to aspects of the present disclosure.

FIG. 6 presents microscopic images of graphene ink formulations highlighting their microstructural characteristics, according to aspects of the present disclosure.

FIG. 7A illustrates an S-parameters graph showing various types of material types, according to aspects of the present disclosure.

FIG. 7B illustrates a plot of film resistivity, according to aspects of the present disclosure.

FIG. 8A through FIG. 8Y depict structured carbons, various carbon nanoparticles, various carbon-containing aggregates, and various three-dimensional carbon-containing structures that are grown over other materials, according to some embodiments of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to sensor technology, specifically to a sensor comprising multiple layers of graphene ink. The sensor, in some embodiments, may be a resonant sensor, a vapor or gas sensor, a biosensor, or a printed label sensor, among others. The sensor may be configured to detect changes in various conditions such as environmental, physical, chemical, biological, electrical, thermal, mechanical, or optical conditions. In an alternative configuration, the sensor may comprise a single layer of graphene ink, and the graphene ink may be configured as a mixture to include two separate types of graphene ink.

In one embodiment, each layer of graphene ink may comprise a mixture of large, conductive patches of graphene and low conductivity interstitial carbon material. In an alternative configuration, a graphene ink mixture, as a single layer, may include a first type of graphene ink configured for conductivity, and a second type of graphene ink configured for wear/tear. The low conductivity interstitial carbon material may serve to bridge the large, conductive patches of graphene, thereby enhancing the sensor's overall conductivity and durability.

In some embodiments, the graphene ink may comprise a first type of graphene configured for conductivity and a second type of graphene configured for wear and tear resistance. As discussed herein, the various types of graphene ink may be configured in a multi-layer configuration (where each layer focuses on a different type of graphene), or be configured to be mixed in a single layer (i.e. multiple types of graphene are combined in a single mixture). As such, these two types of graphene may be mixed together prior to application on the sensor, or they may be applied in separate layers. The order of application (i.e. the layering order) of these two types of graphene may not be integral to the sensor's functionality.

The use of multiple layers of graphene ink in the sensor may provide resistance to wear and tear, potentially extending the sensor's lifespan and improving its performance. This innovative approach to sensor design may offer a solution to the problem of electrical discontinuities caused by cracking in sensors, thereby enhancing the sensor's reliability and effectiveness.

The present disclosure provides a novel and advantageous approach to sensor design, utilizing the properties of multiple types of graphene ink in combination or in layered form to enhance sensor durability and conductivity. This approach may be applicable to a wide range of sensor types and configurations, offering potential benefits in various fields and applications.

### Definitions and Use of Figures

Some of the terms used in this description are defined below for easy reference. The presented terms and their respective definitions are not rigidly restricted to these definitions—a term may be further defined by the term's use within this disclosure. The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application and the appended claims, the term “or” is intended to mean an inclusive “or” rather than an exclusive “or”. That is, unless specified otherwise, or is clear from the context, “X employs A or B” is intended to mean any of the natural inclusive permutations. That is, if X employs A, X employs B, or X employs both A and B, then “X employs A or B” is satisfied under any of the foregoing instances. As used herein, at least one of A or B means at least one of A, or at least one of B, or at least one of both A and B. In other words, this phrase is disjunctive. The articles “a” and “an” as used in this application and the appended claims should generally be construed to mean “one or more” unless specified otherwise or is clear from the context to be directed to a singular form.

Various embodiments are described herein with reference to the figures. It should be noted that the figures are not necessarily drawn to scale, and that elements of similar structures or functions are sometimes represented by like reference characters throughout the figures. It should also be noted that the figures are only intended to facilitate the description of the disclosed embodiments-they are not representative of an exhaustive treatment of all possible embodiments, and they are not intended to impute any limitation as to the scope of the claims. In addition, an illustrated embodiment need not portray all aspects or advantages of usage in any particular environment.

An aspect or an advantage described in conjunction with a particular embodiment is not necessarily limited to that embodiment and can be practiced in any other embodiments even if not so illustrated. References throughout this specification to “some embodiments” or “other embodiments” refer to a particular feature, structure, material or characteristic described in connection with the embodiments as being included in at least one embodiment. Thus, the appearance of the phrases “in some embodiments” or “in other embodiments” in various places throughout this specification are not necessarily referring to the same embodiment or embodiments. The disclosed embodiments are not intended to be limiting of the claims.

### DESCRIPTIONS OF EXEMPLARY EMBODIMENTS

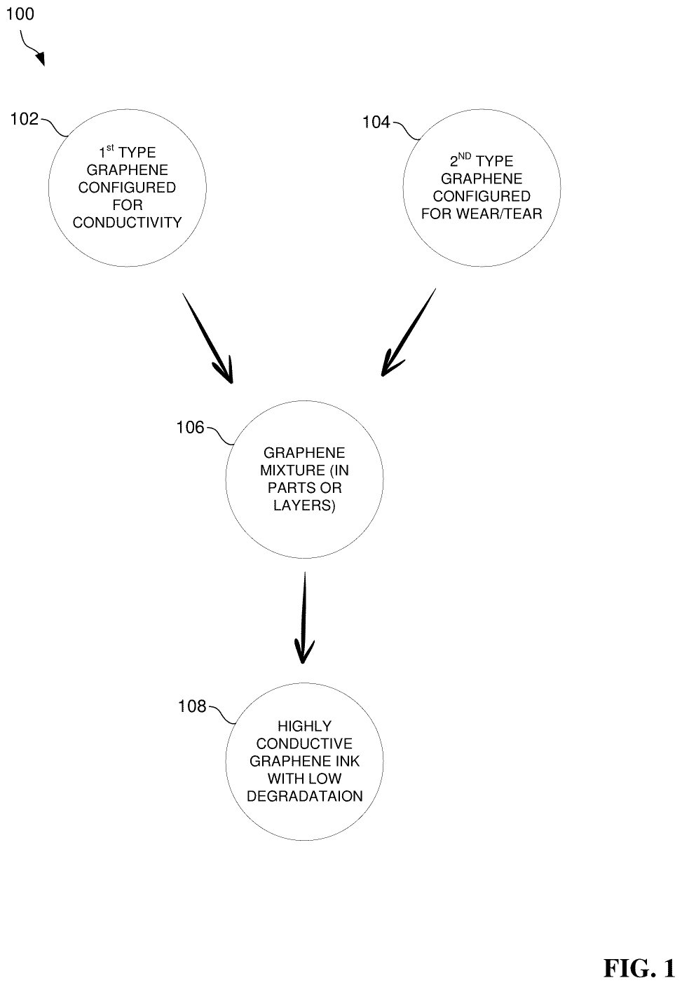

FIG. 1 illustrates a process flowchart 100 for creating a conductive graphene ink with low degradation, in accordance with one embodiment.

Referring to FIG. 1, a process flowchart 100 illustrates the creation of a graphene ink that enhances both conductivity and wear and tear resistance. The process begins with a first type of graphene for conductivity 102 and a second type of graphene for wear and tear resistance 104. In various embodiments, the types of graphene may be achieved via one or more of: graphene concentration, nanoplatelet size distribution, surface functionalization, etc.

In some aspects, these two types of graphene may be combined prior to application on the sensor, forming a graphene mixture in parts or layers 106. For example, in one embodiment, the first type of graphene for conductivity 102 may be applied as a layer, and the second type of graphene for wear and tear resistance 104 may be applied as another layer. In this particular configuration, the benefits of the first type of graphene for conductivity 102 (namely increased conductivity) and the benefits of the second type of graphene for wear and tear resistance 104 (namely increased durability) may be combined via the application of multiple layers. It is recognized that any number of layers may be added, where each layer may, in particular, be configured for a specific benefit. For purposes of clarity, the aggregation of all layers may create a symbiotic benefit (where the benefit of one layer works in conjunction with the benefit of another layer).

In another embodiment, the first type of graphene for conductivity 102 may be combined as a mixture with the second type of graphene for wear and tear resistance 104. The mixture may then be applied as a single layer (or any number of layers), where each layer is a mixture of more than one type of graphene that is preconfigured. For purposes of clarity, the aggregation of all graphene types may create a symbiotic benefit (where the benefit of one type of graphene works in conjunction with the benefit of another type of graphene). Further, it is recognized that any number of types of graphene may be added to the mixture, where each type of graphene may, in particular, be configured for a specific benefit.

Further, the mixture (or application via multiple discrete layers where each layer is configured for a specific benefit) is then processed to produce a final graphene ink with enhanced properties. For example, the result may include highly conductive graphene ink with low degradation 108.

In one embodiment, the first type of graphene for conductivity 102 may be configured to enhance the electrical conductivity of the sensor. Such a graphene may be characterized by its high electron mobility, which allows for efficient charge transport.

In one embodiment, the second type of graphene ink for wear and tear resistance 104 may be configured to enhance the durability of the sensor. Such a graphene may be characterized by its high mechanical strength and flexibility, which can help resist deformation and cracking under stress.

In some cases, the first type of graphene for conductivity 102 and the second type of graphene ink for wear and tear resistance 104 may be mixed together prior to application on the sensor. In one embodiment, this approach may allow for a homogeneous distribution of the two types of graphene within the ink, potentially enhancing the overall performance of the sensor. In other cases, the first type of graphene for conductivity 102 and the second type of graphene ink for wear and tear resistance 104 are applied in separate layers on the sensor. In one embodiment, this approach may allow for a more controlled distribution of the two types of graphene, potentially optimizing the sensor's performance based on the specific requirements of the application. As such, the various types of graphene may be applied via layering (each layer a discrete type of graphene) or a mixture (of more than one type of graphene).

In some embodiments, the first type of graphene for conductivity 102 may be applied before the second type of graphene ink for wear and tear resistance 104. In other embodiments, the second type of graphene ink for wear and tear resistance 104 may be applied before the first type of graphene for conductivity 102. As such, the ordering of the layering may occur in any manner, without any decrease of the benefit of the combination of both layers.

It is recognized that graphene ink may be applied to a variety of types of sensors. For example, in electrochemical sensors, graphene ink may facilitate the detection of chemical and biological analytes. In some instances, these sensors can target specific molecules, such as glucose or DNA, through functionalization with biorecognition elements (such as enzymes and antibodies). Additionally, graphene ink may be used in gas sensors, allowing the detection of gases such as ammonia and nitrogen dioxide through changes in electrical resistance.

Further, graphene ink may be used for real-time monitoring of physiological parameters. For example, strain sensors may be used for monitoring mechanical deformation or in wearable health monitors for tracking vital signs (such as heart rate and body temperature), and water quality or air quality sensors may be used for environmental monitoring (including providing real-time data on pollutants, such as heavy metals in water and volatile organic compounds in the air).

As such, graphene ink may be used by vapor and/or gas sensors (for analyte detection), bio sensors (for enzyme, molecule detection, etc.), resonant sensors (for changes in environment, etc.), and/or any other sensor. Using the disclosure herein would allow for multiple preconfigured benefits that could be used within such sensors, thereby allowing for sensors that can be configured for ensuring electrical continuity and increasing wear and tear resistance (and/or any other preconfigured benefit).

In one embodiment, graphene ink enables high sensitivity and selectivity (thereby detecting minute changes in analyte presence). Notwithstanding such great impact on a variety of markets and sensors, graphene ink-based sensors, especially those used in flexible and wearable devices, are often subjected to repeated mechanical stresses such as bending, stretching, and twisting. Over time, these mechanical deformations can lead to the cracking or delamination of the graphene layers from the substrate, compromising the sensor's performance and reliability.

The disclosure disclosed herein remedies such deficiencies. In particular, using a conventional graphene ink, such ink is often configured for a single benefit (such as conductivity, durability, etc.). In contrast, as disclosed herein, the mixture or layering approach of two different types of graphene ink enables a first benefit in combination with a second benefit (at a minimum). As such, the present disclosure shows that it is possible to achieve one benefit (such as increased conductivity) while also achieve a second benefit (such as increased durability).

More illustrative information will now be set forth regarding various optional architectures and uses in which the foregoing method may or may not be implemented, per the desires of the user. It should be strongly noted that the following information is set forth for illustrative purposes and should not be construed as limiting in any manner. Any of the following features may be optionally incorporated with or without the exclusion of other features described.

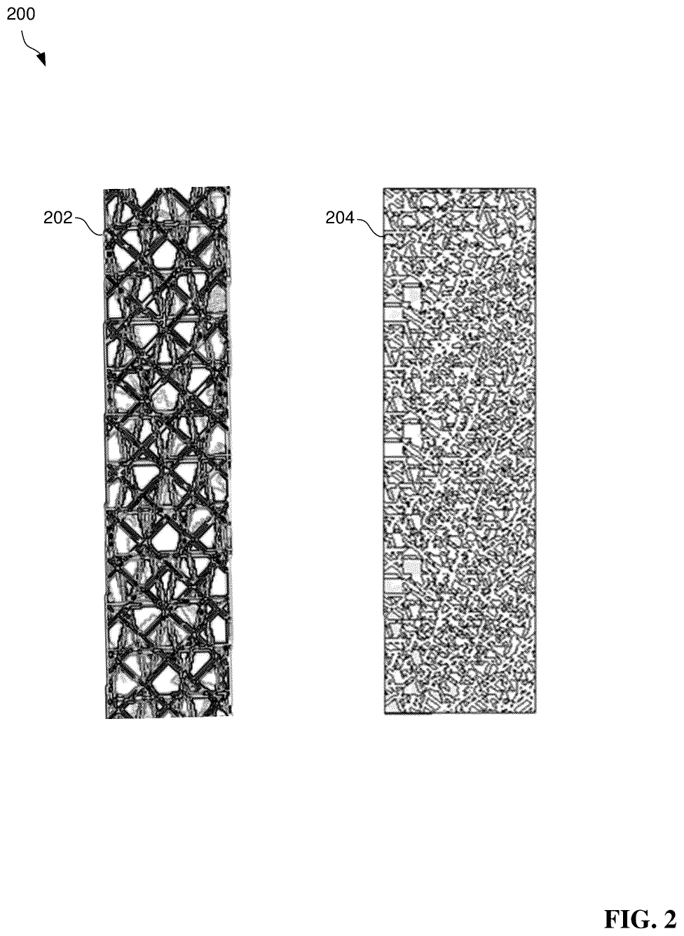

FIG. 2 depicts an orthogonal view 200 of a graphene formation showing distinct configurations. As an option, the orthogonal view 200 may be implemented in the context of any one or more of the embodiments set forth in any previous and/or subsequent figure(s) and/or description thereof. Of course, however, the orthogonal view 200 may be implemented in the context of any desired environment. Further, the aforementioned definitions may equally apply to the description below.

The graphene formation 200 includes showing a large graphene growth 202 and a small graphene growth 204. In particular, the graphene formation 200 shows large islands via the large graphene growth 202 and small islands via the small graphene growth 202.

Additionally, the graphene formation 200 shows the ratio of interstitial space vs graphene island material within the surface area in the sensor (such as a resonator). In one embodiment, the total surface area of the sensor shown is 55.59 mm2. The large graphene growth 202 may correspond with the surface area covered with large islands at 21.39 mm2 (38.5%) with 34.2 mm2 (61.5%) interstitial. Additionally, the small graphene growth 204 may correspond with surface area covered with small islands at 23.21 mm2 (41.75%) with 32.38 mm2 (58.25%) interstitial.

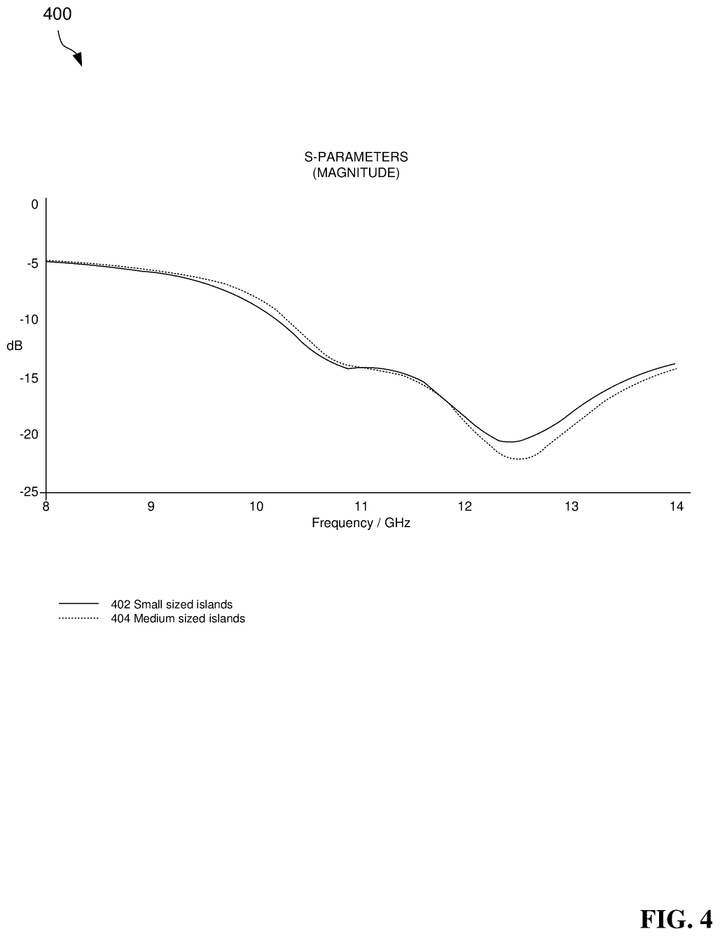

Using the disclosure herein, the graphene ink (comprising of at least two different types of graphene) may be used to achieve a similar level of conductivity while maintaining increased durability. As such, therefore, the size of the graphene islands does not directly affect the combined benefit of the multiple-type configuration of the graphene ink. Such benefit is exemplified herein via FIG. 4 described hereinbelow.

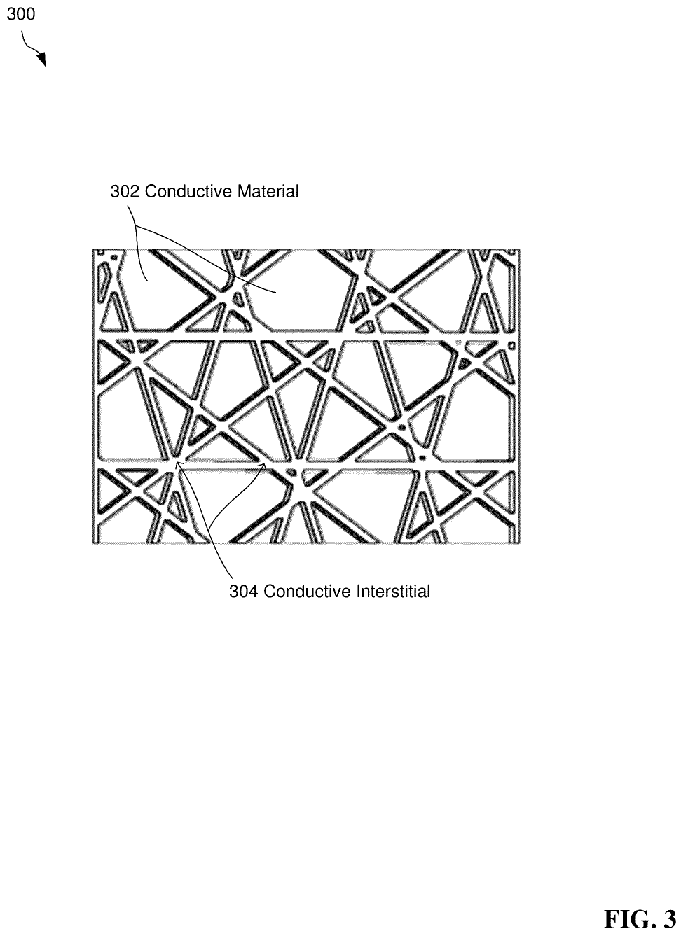

FIG. 3 presents an isometric view 300 of a graphene based material, in accordance with one embodiment. As an option, the isometric view 300 may be implemented in the context of any one or more of the embodiments set forth in any previous and/or subsequent figure(s) and/or description thereof. Of course, however, the isometric view 300 may be implemented in the context of any desired environment. Further, the aforementioned definitions may equally apply to the description below.

As shown, the material growth 300 includes a conductive material 302 that forms a network of interconnected paths. In one embodiment, such interconnected paths may facilitate efficient charge transport and signal transmission. The conductive material 302 may comprise large or small (as shown in FIG. 2), conductive islands of graphene.

Within this network, conductive interstitial 304 are positioned to bridge gaps between the conductive material 302. It is to be appreciated that the conductive interstitial 304 may be the result of formation of the graphene, such as that used for the conductive material 302. In one embodiment, the conductive interstitial 304 may include an insulating zone (such as air) around the conductive material 302.

In conventional graphene ink formulation, the conductive interstitial 304 would often be the location of degradation of the graphene ink (due to breakage between the islands) and form discontinuities. Conversely, using the techniques disclosed herein, the graphene islands may continue to provide high conductivity due to the fact that breakage specifically does not occur (or with reduced occurrence) between the graphene of the conductive material 302.

It is to be appreciated that the conductive interstitial 304 may comprise low conductivity high elastic graphene material, which may serve a dual function in the sensor's graphene ink composition. Firstly, the low conductivity high elastic graphene material may provide a connection between the large, conductive patches of graphene, ensuring continuous electrical conductivity throughout the structure. Secondly, the low conductivity high elastic material may serve to enhance the sensor's durability by resisting deformation and cracking under stress.

In some aspects, the low conductivity high elastic graphene material may be specifically configured to serve as a bridge between the large, conductive patches of graphene of the conductive material 302. Further, the low conductivity high elastic graphene material may include carbon-based filler material. For example, carbon-based filler materials may refer to various carbon forms used to enhance composite materials' properties when incorporated into a matrix. These fillers may include graphene, graphene oxide (GO), reduced graphene oxide (rGO), carbon nanotubes (CNTs), etc.

In various embodiments, incorporating these fillers into matrices may enhance a material's mechanical strength, electrical conductivity, thermal conductivity, and barrier properties.

In some cases, the conductive interstitial 304 may be strategically positioned within the network of interconnected paths formed by the conductive material 302. This arrangement may allow for a non-overlapping pattern of the large, conductive patches of graphene and the low conductivity interstitial carbon material. Such a non-overlapping pattern may enhance the sensor's overall conductivity and durability, as it allows for efficient charge transport across the large, conductive patches of graphene, while also providing a robust bridge for maintaining electrical continuity.

In other cases, the conductive interstitial 304 may be arranged in different configurations within the material growth 300. For instance, the conductive interstitial 304 may be arranged in a layered configuration, a mixed configuration, or any other suitable configuration, depending on the specific requirements of the sensor application. Regardless of the specific configuration, the combination of the conductive patches of graphene and the low conductivity high elastic interstitial carbon material in the graphene ink may provide the sensor with enhanced conductivity and wear and tear resistance.

FIG. 4 shows an S-parameters graph 400 demonstrating the relationship between island size and S-parameter magnitude, in accordance with one embodiment. As an option, the graph 400 may be implemented in the context of any one or more of the embodiments set forth in any previous and/or subsequent figure(s) and/or description thereof. Of course, however, the graph 400 may be implemented in the context of any desired environment. Further, the aforementioned definitions may equally apply to the description below.

As shown, the graph 400 illustrates the magnitude of S-parameters in decibels (dB) across a frequency range in gigahertz (GHz). The small-sized islands line 402 and the medium-sized islands line 404 are plotted on the graph 400, showing their respective S-parameter magnitudes over the frequency range. The small-sized islands line 402 and the medium-sized islands line 404 demonstrate the relationship between island size and S-parameter magnitude.

As discussed herein with reference to FIG. 2, the difference in size of the graphene islands does not significantly affect the S-parameter magnitude, as shown in FIG. 3. In fact, using the two-type graphene composition described herein, the S-parameters magnitude of the small size islands 402 compared to the S-parameters magnitude of the medium size islands 404 are often the same (for example at 8 GHz, both are −5 dB, at 11 GHz, both are about −14 GHZ, etc.). When the S-parameters magnitude diverge (such as at 12.5 GHz), there is not a significant difference in the decibel magnitude (about −20 dB for small sized islands 402 and about −22 dB for medium sized islands 404). Thus, the size of the graphene islands does not significantly affect the conductivity and/or reflectivity of the material when the material comprises two types of graphene.

It is to be appreciated that notwithstanding the similarity in performance of S-parameters magnitude compared to the size of the graphene islands, the graphene islands may be still be preconfigured as desired (large or small). For example, the sensor may be configured to adjust the size of the graphene islands in the graphene ink based on the various desired characteristics.

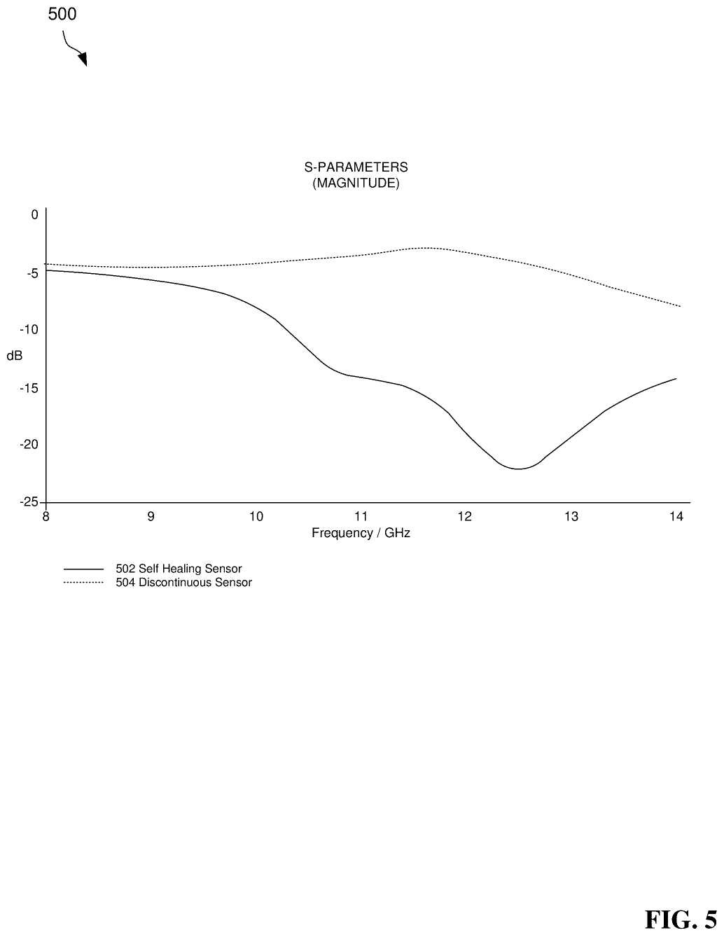

FIG. 5 depicts a graph 500 of S-parameters comparing the performance of a self-healing sensor and a discontinuous sensor, in accordance with one embodiment. As an option, the graph 500 may be implemented in the context of any one or more of the embodiments set forth in any previous and/or subsequent figure(s) and/or description thereof. Of course, however, the graph 500 may be implemented in the context of any desired environment. Further, the aforementioned definitions may equally apply to the description below.

As shown, the graph 500 illustrates the magnitude of S-parameters in decibels (dB) across a frequency range from 8 GHz to 14 GHz. The graph includes a self-healing sensor line 502 and a discontinuous sensor line 504. The self-healing sensor line 502 demonstrates the performance of a resonant sensor with self-healing properties, while the discontinuous sensor line 504 shows the performance of a resonant sensor with discontinuities. In other words, the self healing sensor line 502 may be associated with a sensor configured with at least two-types of graphene type ink, and the discontinuous sensor line 504 may be associated with a sensor configured in a conventional manner (with only a single type of graphene in the graphene ink).

It is to be appreciated that the sensor associated with the self-healing sensor line 502 and/or the discontinuous sensor line 504 may include an analyte sensor, a bio sensor, a resonator sensor, and/or any other type of sensor.

In some aspects, the sensor may be configured to self-heal, meaning that it can recover its functionality after experiencing a disruption, such as a potential crack or deformation.

For example, the graph 500 shows a particular example where the sensor is a resonance sensor, and deformation of the material occurs. As shown, resonance via the self-healing sensor line 502 displays continued function, whereas the discontinuous sensor line 504 shows a lack of function (likely due to discontinuities forming).

As such, material configured with the multiple-type graphene ink composition allows for continued use even after deformation of the material has occurred.

To reemphasize one aspect, the sensor may be configured to minimize the impact of discontinuities on its performance. For example, this can be achieved by using the graphene ink with the mixture of multiple types of preconfigured graphene. The low conductivity high elastic graphene material may serve to bridge the large, conductive patches of graphene, maintaining electrical continuity in the sensor even in the presence of cracks or deformations. As such, this configuration can enhance the performance of the sensor, as represented by the self-healing sensor line 502 on the S-parameters graph 500.

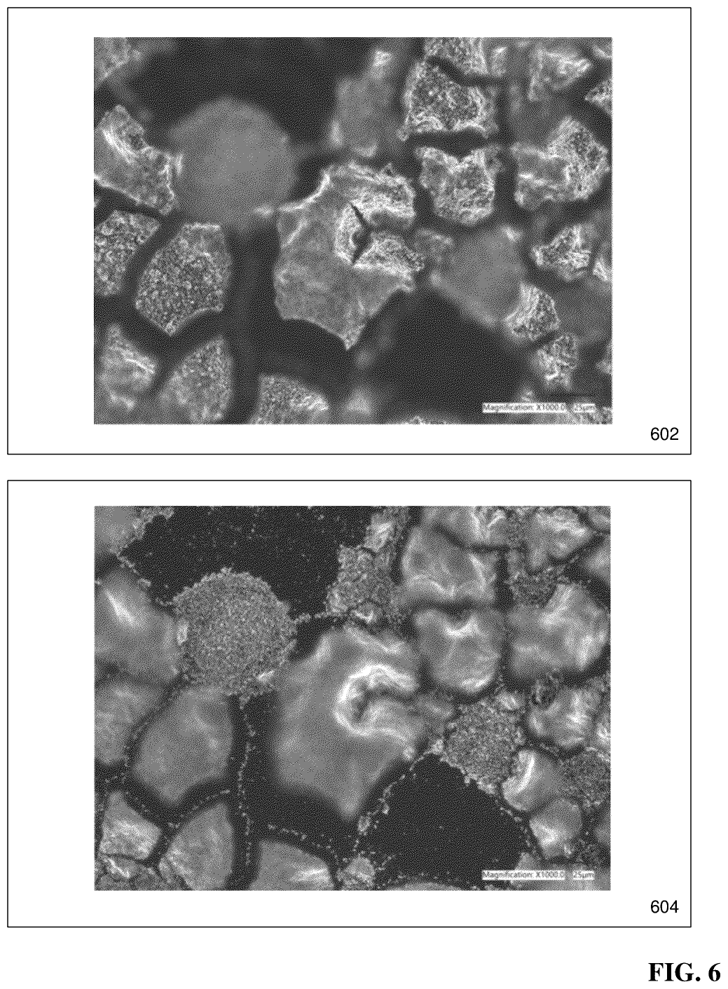

FIG. 6 presents microscopic images of graphene ink formulations highlighting their microstructural characteristics, in accordance with one embodiment. As an option, the images may be implemented in the context of any one or more of the embodiments set forth in any previous and/or subsequent figure(s) and/or description thereof. Of course, however, the images may be implemented in the context of any desired environment. Further, the aforementioned definitions may equally apply to the description below.

As shown, the images provide a detailed view of the microstructural characteristics of the graphene ink. The first focus of graphene ink mixture 602 and the second focus of graphene ink mixture 604 illustrate focus points of the graphene ink.

For example, the first focus of graphene ink mixture 602 shows in focus the graphene islands (corresponding to the conductive material 302). Additionally, the second focus of graphene ink mixture 604 shows in focus the interstitial space (corresponding to the conductive interstitial 304). Again, to note, the image displayed for FIG. 6 is the same for the first focus of graphene ink mixture 602 and the second focus of graphene ink mixture 604 (with altering focus points shown for each).

In one embodiment, the images of FIG. 6 may relate to CO2 reacted carbon nano-onion (CNO). As shown, the second focus of graphene ink mixture 604 exemplifies, in particular, the bridging from one graphene island to another graphene island. Such a bridging serves to not only electrically connect the graphene islands (thereby ensuring conductivity) but to also increase the elasticity of the material.

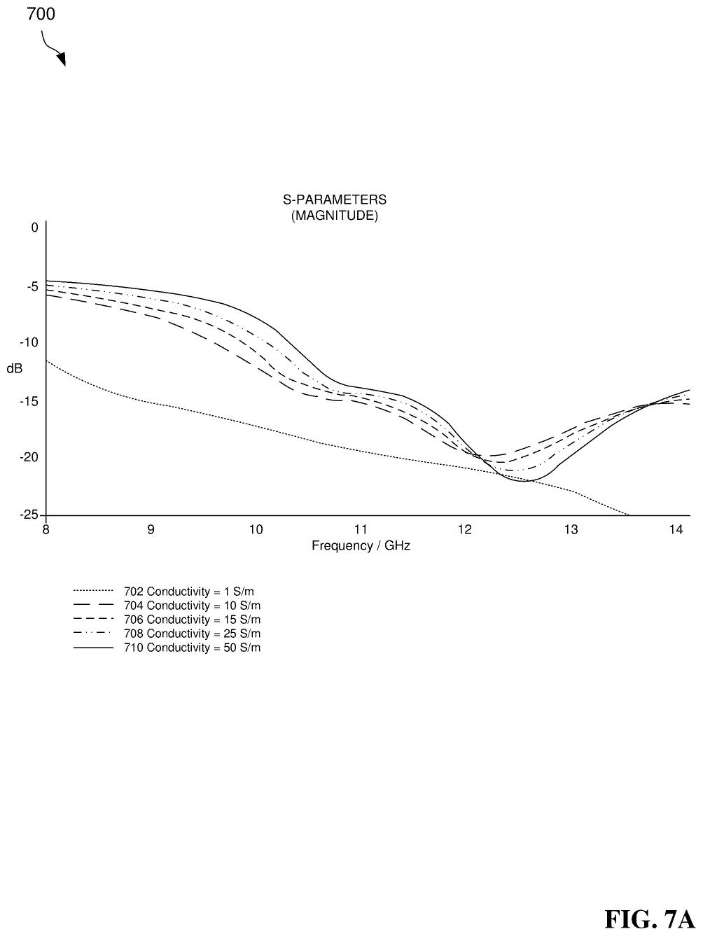

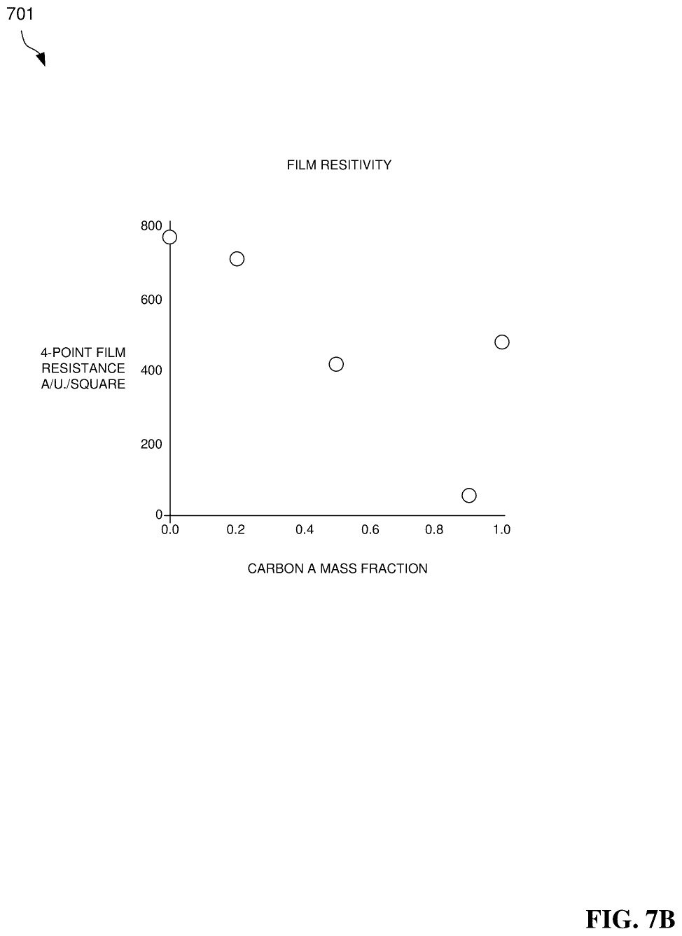

FIG. 7A illustrates an S-parameters graph 700 showing various types of material types, in accordance with one embodiment. As an option, the graph 700 may be implemented in the context of any one or more of the embodiments set forth in any previous and/or subsequent figure(s) and/or description thereof. Of course, however, the graph 700 may be implemented in the context of any desired environment. Further, the aforementioned definitions may equally apply to the description below.

As shown, the graph 700 includes a variety of composite composition types, where each composite composition has a different conductivity value. As such, the graph 700 shows a first line 702 corresponding with a composite conductivity of 1 S/m, a second line 704 corresponding with a composite conductivity of 10 S/m, a third line 706 corresponding with a composite conductivity of 15 S/m, a fourth line 708 corresponding with a composite conductivity of 25 S/m, and a fifth line 710 corresponding with a composite conductivity of 50 S/m. The first line 702 may represent the lowest conductivity value and the fifth line 710 may represent the highest conductivity value.

In various embodiments, the graph 700 may take into account the determination of what happens when composite conductivity decreases. As shown, the lines 704-710 shows a consistent S-parameters magnitude, where as the first line 702 is an anomaly. Such graph 700 shows that the two-type graphene ink construction disclosed herein still functions when the conductivity is greater than 10 S/m. As such, many types of composite composition may benefit from the two-type graphene composition disclosed herein.

In various embodiments, the graphene ink composition disclosed herein may different from conventional graphene ink in several ways. Firstly, the multiple-type graphene ink may use a mixture of different types of graphene ink, or may layer different types of graphene ink. The use of multiple types of graphene ink, each with a different focus and/or benefit, may allow for a more versatile and effective sensor.

Secondly, the multiple-type graphene ink may use low conductivity high elastic graphene ink to bridge the large, conductive islands of high conductivity graphene. The use of low conductivity high elastic graphene ink allows for the maintenance of electrical continuity in the material (and therefore in the sensor), even in the presence of cracks or deformations in the overall graphene ink landscape.

Thirdly, the multiple-type graphene ink sensor can be applied via a resonant sensor, a vapor or gas sensor, a biosensor, a printed label sensor, among others.

In various embodiments, the graphene ink, when layered in multiple coats (each of different graphene type) or applied as a mixture of multiple-types, causes the sensor to increase in wear and tear resistance while maintaining conductivity. In one embodiment, the graphene ink may still crack, but such an effect still does not form large electrical discontinuities.

In one embodiment, a reason for the resistance to cracking may come from the low conductivity high elastic interstitial carbon material that bridges the large, highly conductive patches of graphene. Even if the graphene cracks, the impact of electrical discontinuities is minimal (i.e. the sensor resistance remains high). By contrast, a sensor made entirely of graphene (similar to conventional graphene sensor systems) of similar conductivity to the interstitial carbon would be non-functional. It is the combination of multiple types of graphene (applied via multiple layers or mixed into one layer) that may give rise to the wear and tear resistance property.

It is to be appreciated that the multiple layers of different types of graphene may be applied via conventional curing process. Additionally, the low conductivity interstitial carbon material may be configured based on its non-cracking abilities (configured based on based on particle size, treatment, etc.).

In various embodiments, the disclosure here relates to the concept of segregated print and the goal of having islands of conductive material on/in a lower conductivity crack-free material. Such concept led to the present improvement of disclosed new ink formulation. As such, a segregated/composite print can be achieved by the disclosures herein.

In various embodiments, providing two rates of ink drying in combination with two types of carbon-binder interactions may allow the conductive islands to form without forming electrically insulating cracks in between them.

In various embodiments, the binder-carbon interaction in the ink may perform very different for carbon having a high vs a low surface area.

In various embodiments, the difference in porosity between two types of carbon may affect the binder-carbon powder interaction in the ink.

In various embodiments, there may exist various in BET surface areas between the types of carbon used in the inks selected. For example, in one configuration, more than one order of magnitude difference in BET surface areas (30 to 90 m2/g vs. 800 to 2400 m2/g) may exist between the two types of carbon used in the final ink.

In various embodiments, the drying process and carbon-binder interaction may be very different between the two types of carbons contained in the ink. This configuration may allow for a rearrangement of the carbons into the desired segregated morphology.

In various embodiments, the disclosure may relate to a final sensor trace including a composite material with required precise mechanical and electrical characteristics. Mechanically, the sensor trace may be flexible and remain crack-free. Additionally, the sensor trace may be electrically conductive (or alternatively made of highly electrically conductive elements joined together by a connective material). The connective material may also be electrically conductive. However, in one embodiment, the connective material may not need to be as electrically conductive as the highly electrically conductive material. Such aspects may be configured in a resulting unique ink that allows the print to achieve the necessary features of a radio frequency resonant sensor. Additionally, this disclosure can be applied to RF reflective and RF transmissive sensing applications which require high flexibility (either as the method of sensing or as a mechanism to resist wear and tear).

In various embodiments, an ink may comprise a binder that disperses and carries the ink pigment (e.g. a carbon powder, etc.) to a substrate, a stabilization of the pigment, as well additives dispersion to prevent settling. In one embodiment, the binder may also provide print properties such as ink transfer behavior, setting, and drying characteristics. Despite containing two different types of carbon, the ink may be configured to avoid typical instability problems, flocculation and sedimentation (which can occur with solid particles that have been separated from each other and that are distributed in an ink).

In various embodiments, the system may be configured to provide two different types of carbon contained within the ink in order to achieve the required properties of the print. The binder-carbon interaction may depend on the porosity and structure of the carbon. This binder-carbon interaction may be very different for types of carbon with high surface area versus low surface area.

In various embodiments, more than one order of magnitude difference in BET surface areas (a range of 30 to 90 m2·g-1 vs. a range of 800 to 2400 m2·g-1) between the two types of carbon may be used in the ink. The difference in porosity between the two types of carbon may affect the binder-carbon powder interaction and the drying characteristics of the ink, as an example. The ink drying process, being very different for each of the two types of carbons, may allow for rearrangement of the carbons into the desired segregated composite morphology of the print.

FIG. 7B illustrates a plot 701 of film resistivity, according to aspects of the present disclosure. As an option, the plot 701 may be implemented in the context of any one or more of the embodiments set forth in any previous and/or subsequent figure(s) and/or description thereof. Of course, however, the plot 701 may be implemented in the context of any desired environment. Further, the aforementioned definitions may equally apply to the description below.

As shown, the plot 701 shows a synergistic effect of two specific carbon components. Such plot 701 plots the film resistivity of a print made of various mass ratios of carbon A and carbon B in the ink. The graph shows a deep minimum of film resistivity lower than the resistivity of both films made of pure single carbon material.

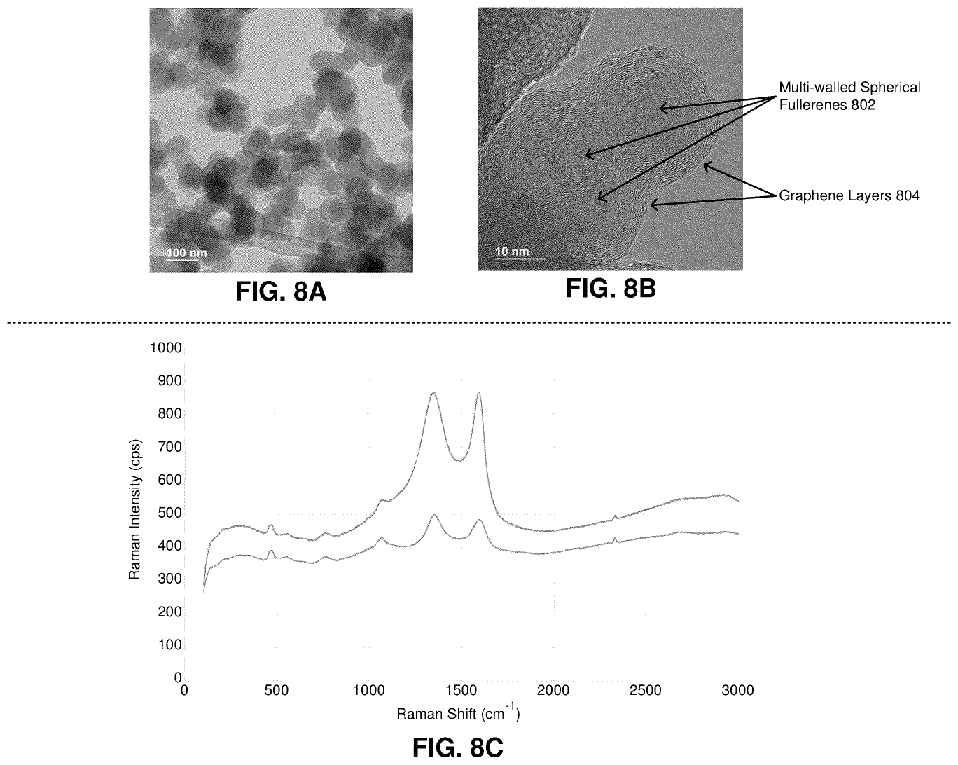

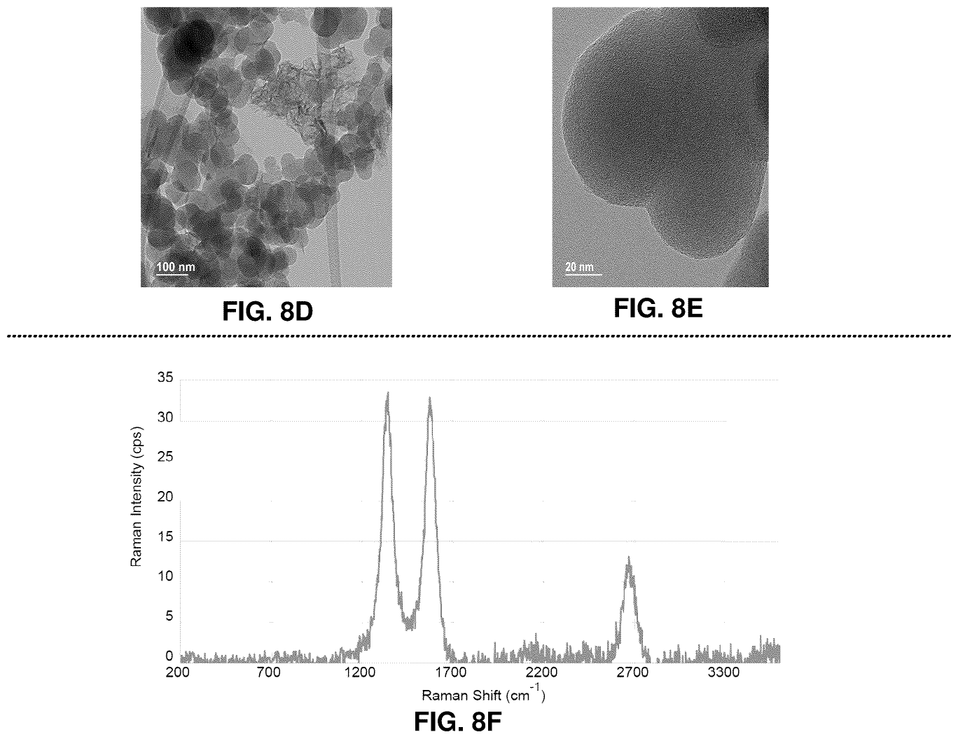

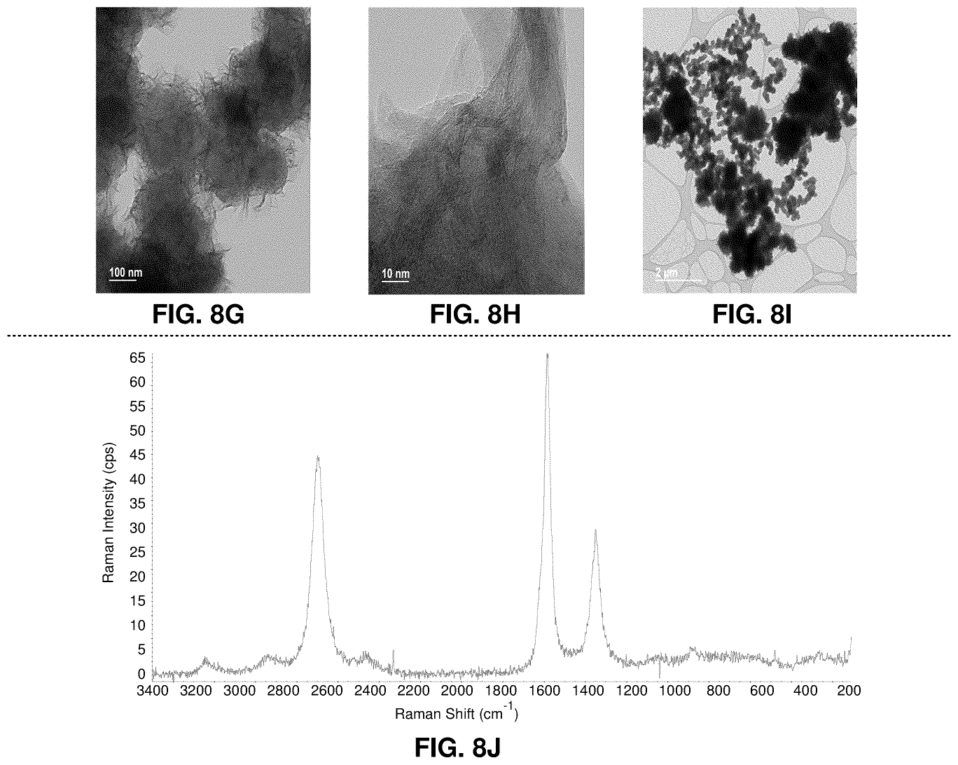

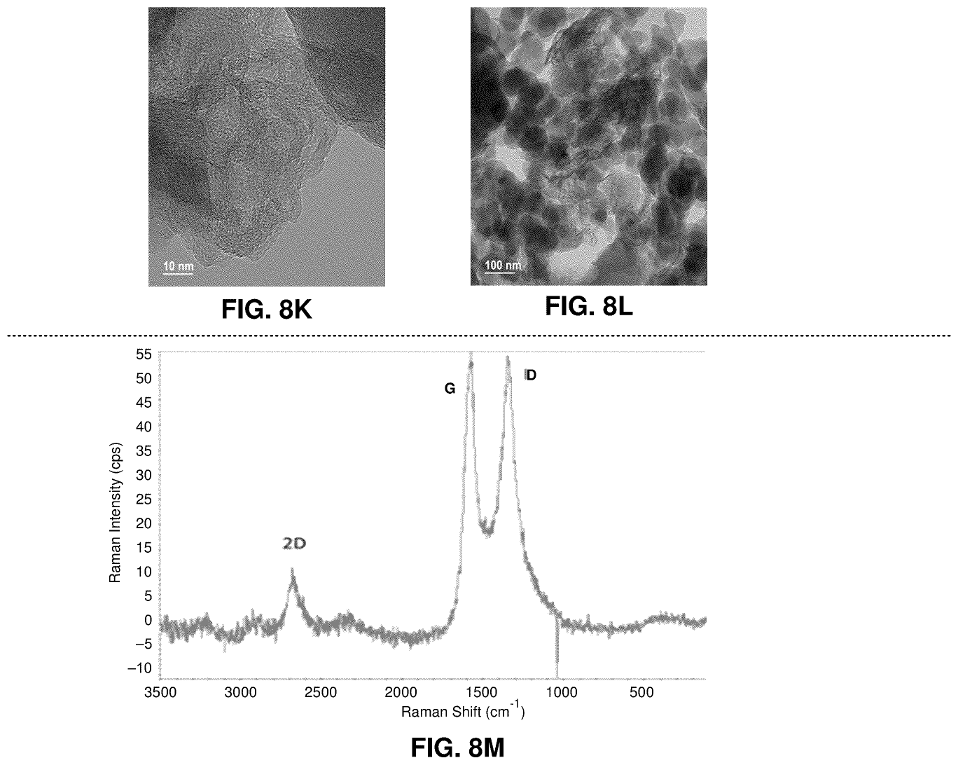

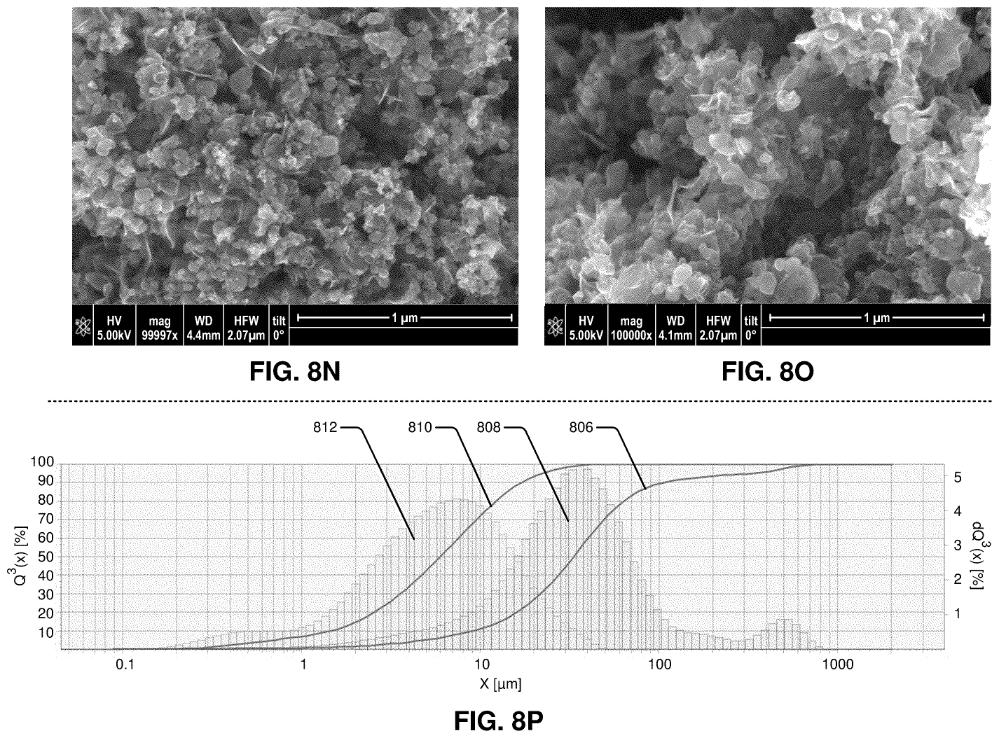

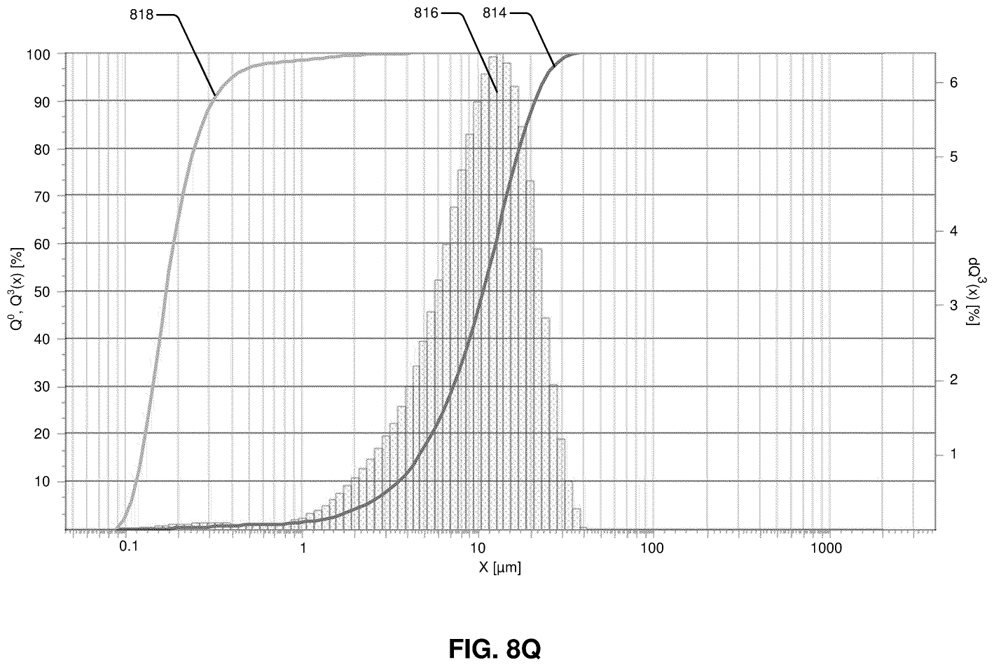

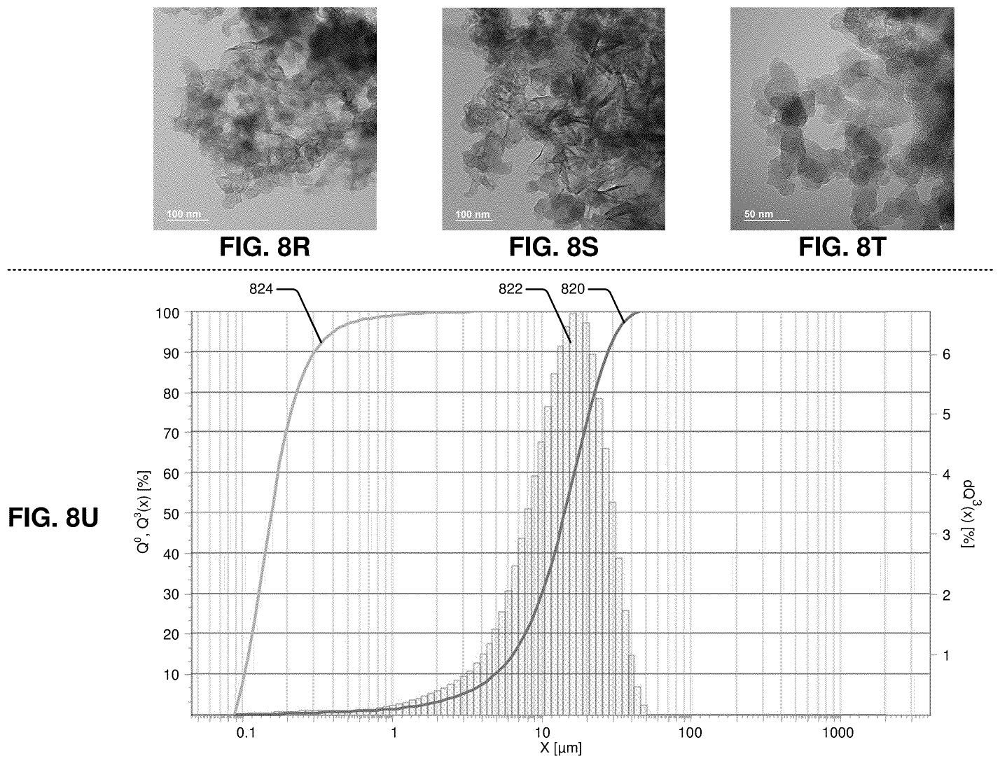

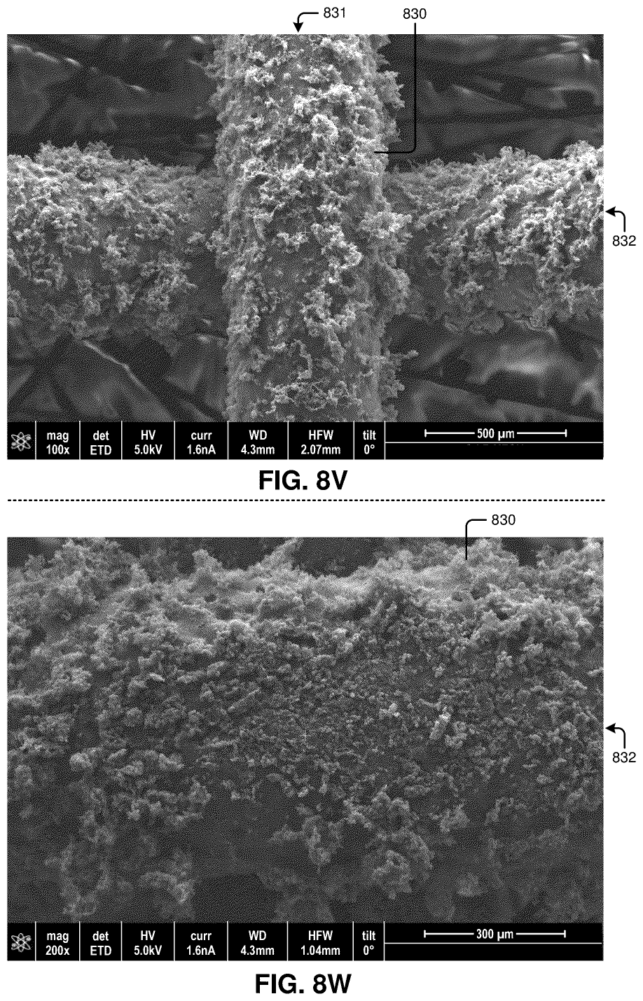

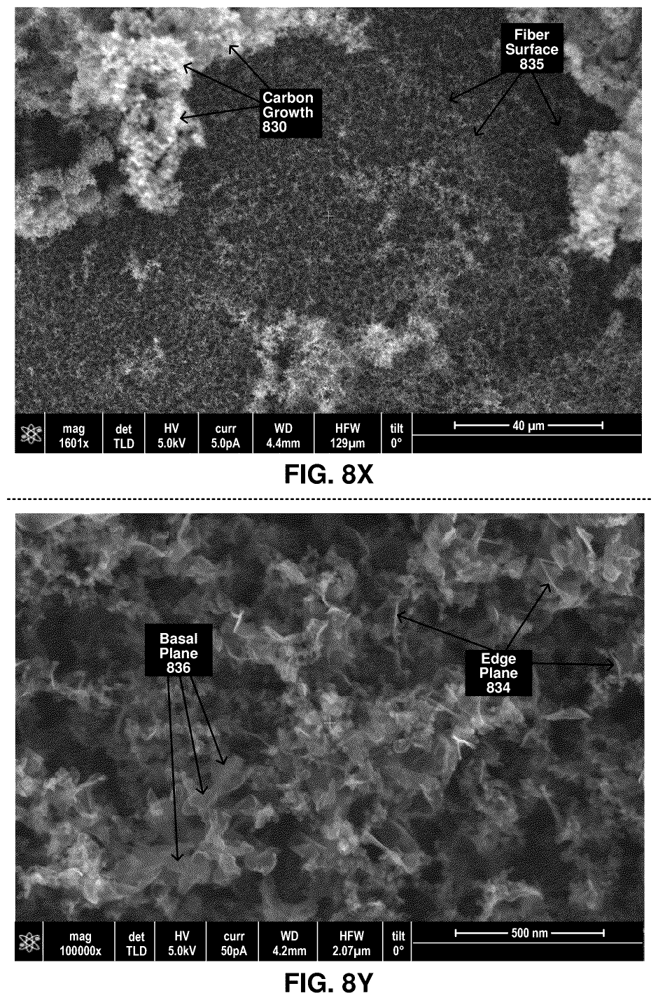

FIG. 8A through FIG. 8Y depict structured carbons, various carbon nanoparticles, various carbon-containing aggregates, and various three-dimensional carbon-containing structures that are grown over other materials, according to some embodiments of the present disclosure.

In some embodiments, the carbon nanoparticles and aggregates are characterized by a high “uniformity” (i.e., high mass fraction of desired carbon allotropes), a high degree of “order” (i.e., low concentration of defects), and/or a high degree of “purity” (i.e., low concentration of elemental impurities), in contrast to the lower uniformity, less ordered, and lower purity particles achievable with conventional systems and methods.

In some embodiments, the nanoparticles produced using the methods described herein contain multi-walled spherical fullerenes (MWSFs) or connected MWSFs and have a high uniformity (e.g., a ratio of graphene to MWSF from 20% to 80%), a high degree of order (e.g., a Raman signature with an ID/IG ratio from 0.95 to 1.05), and a high degree of purity (e.g., the ratio of carbon to other elements (other than hydrogen) is greater than 99.9%). In some embodiments, the nanoparticles produced using the methods described herein contain MWSFs or connected MWSFs, and the MWSFs do not contain a core composed of impurity elements other than carbon. In some cases, the particles produced using the methods described herein are aggregates containing the nanoparticles described above with large diameters (e.g., greater than 10 μm across).

Conventional methods have been used to produce particles containing multi-walled spherical fullerenes with a high degree of order, but the conventional methods lead to carbon products with a variety of shortcomings. For example, high temperature synthesis techniques lead to particles with a mixture of many carbon allotropes and therefore low uniformity (e.g., less than 20% fullerenes to other carbon allotropes) and/or small particle sizes (e.g., less than 1 μm, or less than 100 nm in some cases). Methods using catalysts lead to products including the catalyst elements and therefore have low purity (e.g., less than 95% carbon to other elements) as well. These undesirable properties also often lead to undesirable electrical properties of the resulting carbon particles (e.g., electrical conductivity of less than 1000 S/m).

In some embodiments, the carbon nanoparticles and aggregates described herein are characterized by Raman spectroscopy that is indicative of the high degree of order and uniformity of structure. In some embodiments, the uniform, ordered and/or pure carbon nanoparticles and aggregates described herein are produced using relatively high speed, low cost improved thermal reactors and methods, as described below. Additional advantages and/or improvements will also become apparent from the following disclosure.

In the present disclosure, the term “graphene” refers to an allotrope of carbon in the form of a two-dimensional, atomic-scale, hexagonal lattice in which one atom forms each vertex. The carbon atoms in graphene are sp²-bonded. Additionally, graphene has a Raman spectrum with two main peaks: a G-mode at approximately 1580 cm⁻¹ and a D-mode at approximately 1350 cm⁻¹ (when using a 532 nm excitation laser).

In the present disclosure, the term “fullerene” refers to a molecule of carbon in the form of a hollow sphere, ellipsoid, tube, or other shapes. Spherical fullerenes can also be referred to as Buckminsterfullerenes, or buckyballs. Cylindrical fullerenes can also be referred to as carbon nanotubes. Fullerenes are similar in structure to graphite, which is composed of stacked graphene sheets of linked hexagonal rings. Fullerenes may also contain pentagonal (or sometimes heptagonal) rings.

In the present disclosure, the term “multi-walled fullerene” refers to fullerenes with multiple concentric layers. For example, multi-walled nanotubes (MWNTs) contain multiple rolled layers (concentric tubes) of graphene. Multi-walled spherical fullerenes (MWSFs) contain multiple concentric spheres of fullerenes.

In the present disclosure, the term “nanoparticle” refers to a particle that measures from 1 nm to 999 nm. The nanoparticle can include one or more structural characteristics (e.g., crystal structure, defect concentration, etc.), and one or more types of atoms. The nanoparticle can be any shape, including but not limited to spherical shapes, spheroidal shapes, dumbbell shapes, cylindrical shapes, elongated cylindrical type shapes, rectangular prism shapes, disk shapes, wire shapes, irregular shapes, dense shapes (i.e., with few voids), porous shapes (i.e., with many voids), etc.

In the present disclosure, the term “aggregate” refers to a plurality of nanoparticles that are connected together by electrostatic forces (e.g., Van der Waals forces, London dispersion forces, dipole-dipole interactions, hydrogen bonding, etc.) by covalent bonds, by ionic bonds, by metallic bonds, or by other physical or chemical interactions. Aggregates can vary in size considerably, but in general are larger than about 500 nm.

In some embodiments, a carbon nanoparticle, as described herein, includes two or more connected multi-walled spherical fullerenes (MWSFs) and layers of graphene coating the connected MWSFs. In some embodiments, a carbon nanoparticle, as described herein, includes two or more connected multi-walled spherical fullerenes (MWSFs) and layers of graphene coating the connected MWSFs where the MWSFs do not contain a core composed of impurity elements other than carbon. In some embodiments, a carbon nanoparticle, as described herein, includes two or more connected multi-walled spherical fullerenes (MWSFs) and layers of graphene coating the connected MWSFs where the MWSFs do not contain a void (i.e., a space with no carbon atoms greater than approximately 0.5 nm, or greater than approximately 1 nm) at the center. In some embodiments, the connected MWSFs are formed of concentric, well-ordered spheres of sp²-hybridized carbon atoms, as contrasted with spheres of poorly-ordered, non-uniform, amorphous carbon particles.

In some embodiments, the nanoparticles containing the connected MWSFs have an average diameter in a range from 5 to 500 nm, or from 5 to 250 nm, or from 5 to 100 nm, or from 5 to 50 nm, or from 10 to 500 nm, or from 10 to 250 nm, or from 10 to 100 nm, or from 10 to 50 nm, or from 40 to 500 nm, or from 40 to 250 nm, or from 40 to 100 nm, or from 50 to 500 nm, or from 50 to 250 nm, or from 50 to 100 nm. Of course, nanoparticles containing connected MWSFs may have an average diameter characterized by having any of the foregoing values or being within any of the foregoing exemplary ranges, or an average diameter characterized by having a value or being within a range between any of the foregoing exemplary ranges, without limitation and without departing from the scope of the presently described inventive concepts.

In some embodiments, the carbon nanoparticles described herein form aggregates, wherein many nanoparticles aggregate together to form a larger unit. In some embodiments, a carbon aggregate includes a plurality of carbon nanoparticles. A diameter across the carbon aggregate is in a range from 10 to 500 μm, or from 50 to 500 μm, or from 100 to 500 μm, or from 250 to 500 μm, or from 10 to 250 μm, or from 10 to 100 μm, or from 10 to 50 μm. Of course, carbon aggregates may have an average diameter characterized by having any of the foregoing values or being within any of the foregoing exemplary ranges, or an average diameter characterized by having a value or being within a range between any of the foregoing exemplary ranges, without limitation and without departing from the scope of the presently described inventive concepts.

In some embodiments, the aggregate is formed from a plurality of carbon nanoparticles, as defined above. In some embodiments, aggregates contain connected MWSFs. In some embodiments, the aggregates contain connected MWSFs with a high uniformity metric (e.g., a ratio of graphene to MWSF from 20% to 80%), a high degree of order (e.g., a Raman signature with an I_{D}/I_{G} ratio from 0.95 to 1.05), and a high degree of purity (e.g., greater than 99.9% carbon).

One benefit of producing aggregates of carbon nanoparticles, particularly with diameters in the ranges described above, is that aggregates of particles greater than 10 μm are easier to collect than particles or aggregates of particles that are smaller than 500 nm. The ease of collection reduces the cost of manufacturing equipment used in the production of the carbon nanoparticles and increases the yield of the carbon nanoparticles. Additionally, particles greater than 10 μm in size pose fewer safety concerns compared to the risks of handling smaller nanoparticles, e.g., potential health and safety risks due to inhalation of the smaller nanoparticles. The lower health and safety risks, thus, further reduce the manufacturing cost.

In some embodiments, a carbon nanoparticle has a ratio of graphene to MWSFs from 10% to 90%, or from 10% to 80%, or from 10% to 60%, or from 10% to 40%, or from 10% to 20%, or from 20% to 40%, or from 20% to 90%, or from 40% to 90%, or from 60% to 90%, or from 80% to 90%. In some embodiments, a carbon aggregate has a ratio of graphene to MWSFs is from 10% to 90%, or from 10% to 80%, or from 10% to 60%, or from 10% to 40%, or from 10% to 20%, or from 20% to 40%, or from 20% to 90%, or from 40% to 90%, or from 60% to 90%, or from 80% to 90%. Of course, carbon nanoparticles may have a graphene-to-MWSF ratio characterized by having any of the foregoing values or being within any of the foregoing exemplary ranges, or an average graphene-to-MWSF ratio characterized by having a value or being within a range between any of the foregoing exemplary ranges, without limitation and without departing from the scope of the presently described inventive concepts.

In some embodiments, a carbon nanoparticle has a ratio of graphene to connected MWSFs from 10% to 90%, or from 10% to 80%, or from 10% to 60%, or from 10% to 40%, or from 10% to 20%, or from 20% to 40%, or from 20% to 90%, or from 40% to 90%, or from 60% to 90%, or from 80% to 90%. In some embodiments, a carbon aggregate has a ratio of graphene to connected MWSFs is from 10% to 90%, or from 10% to 80%, or from 10% to 60%, or from 10% to 40%, or from 10% to 20%, or from 20% to 40%, or from 20% to 90%, or from 40% to 90%, or from 60% to 90%, or from 80% to 90%. Of course, carbon nanoparticles may have a graphene-to-connected MWSF ratio characterized by having any of the foregoing values or being within any of the foregoing exemplary ranges, or an average graphene-to-connected MWSF ratio characterized by having a value or being within a range between any of the foregoing exemplary ranges, without limitation and without departing from the scope of the presently described inventive concepts.

In some embodiments, Raman spectroscopy is used to characterize carbon allotropes to distinguish their molecular structures. For example, graphene can be characterized using Raman spectroscopy to determine information such as order/disorder, edge and grain boundaries, thickness, number of layers, doping, strain, and thermal conductivity. MWSFs have also been characterized using Raman spectroscopy to determine the degree of order of the MWSFs.

In some embodiments, Raman spectroscopy is used to characterize the structure of MWSFs or connected MWSFs. The main peaks in the Raman spectra are the G-mode and the D-mode. The G-mode is attributed to the vibration of carbon atoms in sp²-hybridized carbon networks, and the D-mode is related to the breathing of hexagonal carbon rings with defects. In some cases, defects may be present, yet may not be detectable in the Raman spectra. For example, if the presented crystalline structure is orthogonal with respect to the basal plane, the D-peak will show an increase. On the other hand, if presented with a perfectly planar surface that is parallel with respect to the basal plane, the D-peak will be zero.

When using 532 nm incident light, the Raman G-mode is typically at 1582 cm⁻¹ for planar graphite, however can be downshifted for MWSFs or connected MWSFs (e.g., down to 1565 cm⁻¹ or down to 1580 cm⁻¹). The D-mode is observed at approximately 1350 cm⁻¹ in the Raman spectra of MWSFs or connected MWSFs. The ratio of the intensities of the D-mode peak to G-mode peak (i.e., the I_{D}/I_{G}) is related to the degree of order of the MWSFs, where a lower I_{D}/I_{G} indicates a higher degree of order. An I_{D}/I_{G} near or below 1 indicates a relatively high degree of order, and an I_{D}/I_{G} greater than 1.1 indicates a lower degree of order.

In some embodiments, a carbon nanoparticle or a carbon aggregate containing MWSFs or connected MWSFs, as described herein, has a Raman spectrum with a first Raman peak at about 1350 cm⁻¹ and a second Raman peak at about 1580 cm⁻¹ when using 532 nm incident light. In some embodiments, the ratio of an intensity of the first Raman peak to an intensity of the second Raman peak (i.e., the I_{D}/I_{G}) for the nanoparticles or the aggregates described herein is in a range from 0.95 to 1.05, or from 0.9 to 1.1, or from 0.8 to 1.2, or from 0.9 to 1.2, or from 0.8 to 1.1, or from 0.5 to 1.5, or less than 1.5, or less than 1.2, or less than 1.1, or less than 1, or less than 0.95, or less than 0.9, or less than 0.8. Of course, carbon nanoparticles or aggregates including MWSFs or connected MWSFs may be characterized by a ratio of first and second Raman peak intensities having any of the foregoing values or being within any of the foregoing exemplary ranges, or a ratio of first and second Raman peak intensities characterized by having a value or being within a range between any of the foregoing exemplary ranges, without limitation and without departing from the scope of the presently described inventive concepts.

In some embodiments, a carbon aggregate containing MWSFs or connected MWSFs, as defined above, has a high purity. In some embodiments, the carbon aggregate containing MWSFs or connected MWSFs has a ratio of carbon to metals of greater than 99.99%, or greater than 99.95%, or greater than 99.9%, or greater than 99.8%, or greater than 99.5%, or greater than 99%. In some embodiments, the carbon aggregate has a ratio of carbon to other elements of greater than 99.99%, or greater than 99.95%, or greater than 99.9%, or greater than 99.5%, or greater than 99%, or greater than 90%, or greater than 80%, or greater than 70%, or greater than 60%. In some embodiments, the carbon aggregate has a ratio of carbon to other elements (except for hydrogen) of greater than 99.99%, or greater than 99.95%, or greater than 99.9%, or greater than 99.8%, or greater than 99.5%, or greater than 99%, or greater than 90%, or greater than 80%, or greater than 70%, or greater than 60%. Of course, carbon aggregates including MWSFs or connected MWSFs may be characterized by a ratio of carbon to metal having any of the foregoing values or being within any of the foregoing exemplary ranges, or a ratio of carbon to metal having value or being within a range between any of the foregoing exemplary ranges, without limitation and without departing from the scope of the presently described inventive concepts.

In some embodiments, a carbon aggregate containing MWSFs or connected MWSFs, as defined above, has a high specific surface area. In some embodiments, the carbon aggregate has a Brunauer, Emmett and Teller (BET) specific surface area from 10 to 200 m²/g, or from 10 to 100 m²/g, or from 10 to 50 m2/g, or from 50 to 200 m²/g, or from 50 to 100 m²/g, or from 10 to 1000 m²/g. Of course, carbon aggregates including MWSFs or connected MWSFs may be characterized by a BET specific surface area having any of the foregoing values or being within any of the foregoing exemplary ranges, or a BET specific surface area characterized by having a value or being within a range between any of the foregoing exemplary ranges, without limitation and without departing from the scope of the presently described inventive concepts.

In some embodiments, a carbon aggregate containing MWSFs or connected MWSFs, as defined above, has a high electrical conductivity. In some embodiments, a carbon aggregate containing MWSFs or connected MWSFs, as defined above, is compressed into a pellet and the pellet has an electrical conductivity greater than 500 S/m, or greater than 1000 S/m, or greater than 2000 S/m, or greater than 3000 S/m, or greater than 4000 S/m, or greater than 5000 S/m, or greater than 10000 S/m, or greater than 20000 S/m, or greater than 30000 S/m, or greater than 40000 S/m, or greater than 50000 S/m, or greater than 60000 S/m, or greater than 70000 S/m, or from 500 S/m to 100000 S/m, or from 500 S/m to 1000 S/m, or from 500 S/m to 10000 S/m, or from 500 S/m to 20000 S/m, or from 500 S/m to 100000 S/m, or from 1000 S/m to 10000 S/m, or from 1000 S/m to 20000 S/m, or from 10000 to 100000 S/m, or from 10000 S/m to 80000 S/m, or from 500 S/m to 10000 S/m. Of course, carbon aggregates including MWSFs or connected MWSFs may be characterized by an electrical conductivity having any of the foregoing values or being within any of the foregoing exemplary ranges, or an electrical conductivity characterized by having a value or being within a range between any of the foregoing exemplary ranges, without limitation and without departing from the scope of the presently described inventive concepts.

In some cases, the density of the pellet is approximately 1 g/cm³, or approximately 1.2 g/cm³, or approximately 1.5 g/cm³, or approximately 2 g/cm³, or approximately 2.2 g/cm³, or approximately 2.5 g/cm³, or approximately 3 g/cm³. Of course, pellets may be characterized by a density having any of the foregoing values or being within any of the foregoing exemplary ranges, or a density having a value or being within a range between any of the foregoing exemplary ranges, without limitation and without departing from the scope of the presently described inventive concepts.

Additionally, tests have been performed in which compressed pellets of the carbon aggregate materials have been formed with compressions of 2000 psi and 12000 psi and with annealing temperatures of 800° C. and 1000° C. The higher compression and/or the higher annealing temperatures generally result in pellets with a higher degree of electrical conductivity, including in the range of 12410.0 S/m to 13173.3 S/m.

### High Purity Carbon Allotropes Produced Using Thermal Processing Systems

In some embodiments, the carbon nanoparticles and aggregates described herein are produced using thermal reactors and methods, such as any appropriate thermal reactor and/or method. Further details pertaining to thermal reactors and/or methods of use can be found in U.S. Pat. No. 9,862,602, issued Jan. 9, 2018, titled “CRACKING OF A PROCESS GAS”, which is hereby incorporated by reference in its entirety. Additionally, precursors (e.g., including methane, ethane, propane, butane, and natural gas) can be used with the thermal reactors to produce the carbon nanoparticles and the carbon aggregates described herein.

In some embodiments, the carbon nanoparticles and aggregates described herein are produced using the thermal reactors with gas flow rates from 1 slm to 10 slm, or from 0.1 slm to 20 slm, or from 1 slm to 5 slm, or from 5 slm to 10 slm, or greater than 1 slm, or greater than 5 slm. In some embodiments, the carbon nanoparticles and aggregates described herein are produced using the thermal reactors with gas resonance times from 0.1 seconds to 30 seconds, or from 0.1 seconds to 10 seconds, or from 1 seconds to 10 seconds, or from 1 seconds to 5 seconds, from 5 seconds to 10 seconds, or greater than 0.1 seconds, or greater than 1 seconds, or greater than 5 seconds, or less than 30 seconds. Of course, carbon nanoparticles and aggregates may be produced using thermal reactors with gas flow rates having any of the foregoing values or being within any of the foregoing exemplary ranges, or gas flow rates having a value or being within a range between any of the foregoing exemplary ranges, without limitation and without departing from the scope of the presently described inventive concepts.

In some embodiments, the carbon nanoparticles and aggregates described herein are produced using the thermal reactors with production rates from 10 g/hr to 200 g/hr, or from 30 g/hr to 200 g/hr, or from 30 g/hr to 100 g/hr, or from 30 g/hr to 60 g/hr, or from 10 g/hr to 100 g/hr, or greater than 10 g/hr, or greater than 30 g/hr, or greater than 100 g/hr. Of course, carbon nanoparticles and aggregates may be produced using thermal reactors with production rates having any of the foregoing values or being within any of the foregoing exemplary ranges, or production rates having a value or being within a range between any of the foregoing exemplary ranges, without limitation and without departing from the scope of the presently described inventive concepts.

In some embodiments, thermal reactors or other cracking apparatuses and thermal reactor methods or other cracking methods can be used for refining, pyrolyzing, dissociating or cracking feedstock process gases into its constituents to produce the carbon nanoparticles and the carbon aggregates described herein, as well as other solid and/or gaseous products (e.g., hydrogen gas and/or lower order hydrocarbon gases). The feedstock process gases generally include, for example, hydrogen gas (H₂), carbon dioxide (CO₂), C₁ to C₁₀ hydrocarbons, aromatic hydrocarbons, and/or other hydrocarbon gases such as natural gas, methane, ethane, propane, butane, isobutane, saturated/unsaturated hydrocarbon gases, ethene, propene, etc., and mixtures thereof. The carbon nanoparticles and the carbon aggregates can include, for example, multi-walled spherical fullerenes (MWSFs), connected MWSFs, carbon nanospheres, graphene, graphite, highly ordered pyrolytic graphite, single-walled nanotubes, multi-walled nanotubes, other solid carbon products, and/or the carbon nanoparticles and the carbon aggregates described herein.

Some embodiments for producing the carbon nanoparticles and the carbon aggregates described herein include thermal cracking methods that use, for example, an elongated longitudinal heating element optionally enclosed within an elongated casing, housing or body of a thermal cracking apparatus. The body generally includes, for example, one or more tubes or other appropriate enclosures made of stainless steel, titanium, graphite, quartz, or the like. In some embodiments, the body of the thermal cracking apparatus is generally cylindrical in shape with a central elongate longitudinal axis arranged vertically and a feedstock process gas inlet at or near a top of the body. The feedstock process gas flows longitudinally down through the body or a portion thereof. In the vertical configuration, both gas flow and gravity assist in the removal of the solid products from the body of the thermal cracking apparatus.

The heating element generally includes, for example, a heating lamp, one or more resistive wires or filaments (or twisted wires), metal filaments, metallic strips or rods, and/or other appropriate thermal radical generators or elements that can be heated to a specific temperature (i.e., a molecular cracking temperature) sufficient to thermally crack molecules of the feedstock process gas. The heating element is generally disposed, located or arranged to extend centrally within the body of the thermal cracking apparatus along the central longitudinal axis thereof. For example, if there is only one heating element, then it is placed at or concentric with the central longitudinal axis, and if there is a plurality of the heating elements, then they are spaced or offset generally symmetrically or concentrically at locations near and around and parallel to the central longitudinal axis.

Thermal cracking to produce the carbon nanoparticles and aggregates described herein is generally achieved by passing the feedstock process gas over, or in contact with, or within the vicinity of, the heating element within a longitudinal elongated reaction zone generated by heat from the heating element and defined by and contained inside the body of the thermal cracking apparatus to heat the feedstock process gas to or at a specific molecular cracking temperature.

The reaction zone is considered to be the region surrounding the heating element and close enough to the heating element for the feedstock process gas to receive sufficient heat to thermally crack the molecules thereof. The reaction zone is thus generally axially aligned or concentric with the central longitudinal axis of the body. In some embodiments, the thermal cracking is performed under a specific pressure. In some embodiments, the feedstock process gas is circulated around or across the outside surface of a container of the reaction zone or a heating chamber in order to cool the container or chamber and preheat the feedstock process gas before flowing the feedstock process gas into the reaction zone.

In some embodiments, the carbon nanoparticles and aggregates described herein and/or hydrogen gas are produced without the use of catalysts. In other words, the process is catalyst free.

Some embodiments to produce the carbon nanoparticles and aggregates described herein using thermal cracking apparatuses and methods to provide a standalone system that can advantageously be rapidly scaled up or scaled down for different production levels as desired. For example, some embodiments are scalable to provide a standalone hydrogen and/or carbon nanoparticle producing station, a hydrocarbon source, or a fuel cell station. Some embodiments can be scaled up to provide higher capacity systems, e.g., for a refinery or the like.

In some embodiments, a thermal cracking apparatus for cracking a feedstock process gas to produce the carbon nanoparticles and aggregates described herein include a body, a feedstock process gas inlet, and an elongated heating element. The body has an inner volume with a longitudinal axis. The inner volume has a reaction zone concentric with the longitudinal axis. A feedstock process gas is flowed into the inner volume through the feedstock process gas inlet during thermal cracking operations. The elongated heating element is disposed within the inner volume along the longitudinal axis and is surrounded by the reaction zone. During the thermal cracking operations, the elongated heating element is heated by electrical power to a molecular cracking temperature to generate the reaction zone, the feedstock process gas is heated by heat from the elongated heating element, and the heat thermally cracks molecules of the feedstock process gas that are within the reaction zone into constituents of the molecules.

In some embodiments, a method for cracking a feedstock process gas to produce the carbon nanoparticles and aggregates described herein includes: (1) providing a thermal cracking apparatus having an inner volume that has a longitudinal axis and an elongated heating element disposed within the inner volume along the longitudinal axis; (2) heating the elongated heating element by electrical power to a molecular cracking temperature to generate a longitudinal elongated reaction zone within the inner volume; (3) flowing a feedstock process gas into the inner volume and through the longitudinal elongated reaction zone (e.g., wherein the feedstock process gas is heated by heat from the elongated heating element); and (4) thermally cracking molecules of the feedstock process gas within the longitudinal elongated reaction zone into constituents thereof (e.g., hydrogen gas and one or more solid products) as the feedstock process gas flows through the longitudinal elongated reaction zone.

In some embodiments, the feedstock process gas to produce the carbon nanoparticles and aggregates described herein includes a hydrocarbon gas. The results of cracking include hydrogen (e.g., H²) and various forms of the carbon nanoparticles and aggregates described herein. In some embodiments, the carbon nanoparticles and aggregates include two or more MWSFs and layers of graphene coating the MWSFs, and/or connected MWSFs and layers of graphene coating the connected MWSFs. In some embodiments, the feedstock process gas is preheated (e.g., to 100° C. to 500° C.) by flowing the feedstock process gas through a gas preheating region between a heating chamber and a shell of the thermal cracking apparatus before flowing the feedstock process gas into the inner volume. In some embodiments, a gas having nanoparticles therein is flowed into the inner volume and through the longitudinal elongated reaction zone to mix with the feedstock process gas, and a coating of a solid product (e.g., layers of graphene) is formed around the nanoparticles.

### Post-Processing High Purity Structured Carbons

In some embodiments, the carbon nanoparticles and aggregates containing multi-walled spherical fullerenes (MWSFs) or connected MWSFs described herein are produced and collected, and no post-processing is done. In other embodiments, the carbon nanoparticles and aggregates containing multi-walled spherical fullerenes (MWSFs) or connected MWSFs described herein are produced and collected, and some post-processing is done. Some examples of post-processing include mechanical processing such as ball milling, grinding, attrition milling, micro fluidizing, and other techniques to reduce the particle size without damaging the MWSFs. Some further examples of post-processing include exfoliation processes such as sheer mixing, chemical etching, oxidizing (e.g., Hummers' method), thermal annealing, doping by adding elements during annealing (e.g., sulfur, nitrogen), steaming, filtering, and lyophilizing, among others. Some examples of post-processing include sintering processes such as spark plasma sintering (SPS), direct current sintering, microwave sintering, and ultraviolet (UV) sintering, which can be conducted at high pressure and temperature in an inert gas. In some embodiments, multiple post-processing methods can be used together or in a series. In some embodiments, the post-processing produces functionalized carbon nanoparticles or aggregates containing multi-walled spherical fullerenes (MWSFs) or connected MWSFs.

In some embodiments, the materials are mixed together in different combinations. In some embodiments, different carbon nanoparticles and aggregates containing MWSFs or connected MWSFs described herein are mixed together before post-processing. For example, different carbon nanoparticles and aggregates containing MWSFs or connected MWSFs with different properties (e.g., different sizes, different compositions, different purities, from different processing runs, etc.) can be mixed together. In some embodiments, the carbon nanoparticles and aggregates containing MWSFs or connected MWSFs described herein can be mixed with graphene to change the ratio of the connected MWSFs to graphene in the mixture. In some embodiments, different carbon nanoparticles and aggregates containing MWSFs or connected MWSFs described herein can be mixed together after post-processing. For example, different carbon nanoparticles and aggregates containing MWSFs or connected MWSFs with different properties and/or different post-processing methods (e.g., different sizes, different compositions, different functionality, different surface properties, different surface areas) can be mixed together.

In some embodiments, the carbon nanoparticles and aggregates described herein are produced and collected, and subsequently processed by mechanical grinding, milling, and/or exfoliating. In some embodiments, the processing (e.g., by mechanical grinding, milling, exfoliating, etc.) reduces the average size of the particles. In some embodiments, the processing (e.g., by mechanical grinding, milling, exfoliating, etc.) increases the average surface area of the particles. In some embodiments, the processing by mechanical grinding, milling and/or exfoliation shears off some fraction of the carbon layers, producing sheets of graphite mixed with the carbon nanoparticles.

In some embodiments, the mechanical grinding or milling is performed using a ball mill, a planetary mill, a rod mill, a shear mixer, a high-shear granulator, an autogenous mill, or other types of machining used to break solid materials into smaller pieces by grinding, crushing or cutting. In some embodiments, the mechanical grinding, milling and/or exfoliating is performed wet or dry. In some embodiments, the mechanical grinding is performed by grinding for some period of time, then idling for some period of time, and repeating the grinding and idling for a number of cycles. In some embodiments, the grinding period is from 1 minute to 20 minutes, or from 1 minute to 10 minutes, or from 3 minutes to 8 minutes, or approximately 3 minutes, or approximately 8 minutes. In some embodiments, the idling period is from 1 minute to 10 minutes, or approximately 5 minutes, or approximately 6 minutes. In some embodiments, the number of grinding and idling cycles is from 1 minute to 100 minutes, or from 5 minutes to 100 minutes, or from 10 minutes to 100 minutes, or from 5 minutes to 10 minutes, or from 5 minutes to 20 minutes. In some embodiments, the total amount of time of grinding and idling is from 10 minutes to 1200 minutes, or from 10 minutes to 600 minutes, or from 10 minutes to 240 minutes, or from 10 minutes to 120 minutes, or from 100 minutes to 90 minutes, or from 10 minutes to 60 minutes, or approximately 90 minutes, or approximately 120 minutes. Of course, grinding, milling, or idling times within the scope of the presently disclosed inventive embodiments may have any of the foregoing values or be within any of the foregoing exemplary ranges, between any of the foregoing exemplary ranges, without limitation and without departing from the scope of the presently described inventive concepts.

In some embodiments, the grinding steps in the cycle are performed by rotating a mill in one direction for a first cycle (e.g., clockwise), and then rotating a mill in the opposite direction (e.g., counterclockwise) for the next cycle. In some embodiments, the mechanical grinding or milling is performed using a ball mill, and the grinding steps are performed using a rotation speed from 100 to 1000 rpm, or from 100 to 500 rpm, or approximately 400 rpm, or any value or range of values therebetween. In some embodiments, the mechanical grinding or milling is performed using a ball mill that uses a milling media with a diameter from 0.1 mm to 20 mm, or from 0.1 mm to 10 mm, or from 1 mm to 10 mm, or approximately 0.1 mm, or approximately 1 mm, or approximately 10 mm, or any value or range of values therebetween. In some embodiments, the mechanical grinding or milling is performed using a ball mill that uses a milling media composed of metal such as steel, an oxide such as zirconium oxide (zirconia), yttria stabilized zirconium oxide, silica, alumina, magnesium oxide, or other hard materials such as silicon carbide or tungsten carbide.

In some embodiments, the carbon nanoparticles and aggregates described herein are produced and collected, and subsequently processed using elevated temperatures such as thermal annealing or sintering. In some embodiments, the processing using elevated temperatures is done in an inert environment such as nitrogen or argon. In some embodiments, the processing using elevated temperatures is done at atmospheric pressure, or under vacuum, or at low pressure. In some embodiments, the processing using elevated temperatures is done at a temperature from 500° C. to 2500° C., or from 500° C. to 1500° C., or from 800° C. to 1500° C., or from 800° C. to 1200° C., or from 800° C. to 1000° C., or from 2000° C. to 2400° C., or approximately 800° C., or approximately 1000° C., or approximately 1500° C., or approximately 2000° C., or approximately 2400° C. Of course, processing using elevated temperatures may be performed at any of the foregoing temperatures, or at a temperature within any of the foregoing exemplary ranges, or between any of the foregoing exemplary ranges, without limitation and without departing from the scope of the presently described inventive concepts.

In some embodiments, the carbon nanoparticles and aggregates described herein are produced and collected, and subsequently, in post processing steps, additional elements or compounds are added to the carbon nanoparticles, thereby incorporating the unique properties of the carbon nanoparticles and aggregates into other mixtures of materials.

In some embodiments, either before or after post-processing, the carbon nanoparticles and aggregates described herein are added to solids, liquids or slurries of other elements or compounds to form additional mixtures of materials incorporating the unique properties of the carbon nanoparticles and aggregates. In some embodiments, the carbon nanoparticles and aggregates described herein are mixed with other solid particles, polymers or other materials.

In some embodiments, either before or after post-processing, the carbon nanoparticles and aggregates described herein are used in various applications beyond applications pertaining to the present application. Such applications including but not limited to transportation applications (e.g., automobile and truck tires, couplings, mounts, elastomeric o-rings, hoses, sealants, grommets, etc.) and industrial applications (e.g., rubber additives, functionalized additives for polymeric materials, additives for epoxies, etc.).

FIGS. 8A and 8B show transmission electron microscope (TEM) images of as-synthesized carbon nanoparticles. The carbon nanoparticles of FIG. 8A (at a first magnification) and FIG. 8B (at a second magnification) contain connected multi-walled spherical fullerenes 802 (MWSFs) with graphene layers 804 that coat the connected MWSFs. The ratio of MWSF to graphene allotropes in this example is approximately 80% due to the relatively short resonance times. The MWSFs in FIG. 8A are approximately 5 nm to 10 nm in diameter, and the diameter can be from 5 nm to 500 nm using the conditions described above. In some embodiments, the average diameter across the MWSFs is in a range from 5 nm to 500 nm, or from 5 nm to 250 nm, or from 5 nm to 100 nm, or from 5 nm to 50 nm, or from 10 nm to 500 nm, or from 10 nm to 250 nm, or from 10 nm to 100 nm, or from 10 nm to 50 nm, or from 40 nm to 500 nm, or from 40 nm to 250 nm, or from 40 nm to 100 nm, or from 50 nm to 500 nm, or from 50 nm to 250 nm, or from 50 nm to 100 nm. Of course, average MWSF diameter within the scope of the presently disclosed inventive embodiments may have any of the foregoing values or be within any of the foregoing exemplary ranges, or between any of the foregoing exemplary ranges, without limitation and without departing from the scope of the presently described inventive concepts. No catalyst was used in this process, and therefore, there is no central seed containing contaminants. The aggregate particles produced in this example had a particle size of approximately 10 μm to 100 μm, or approximately 10 μm to 500 μm.

FIG. 8C shows the Raman spectrum of the as-synthesized aggregates in this example taken with 532 nm incident light. The I_{D}/I_{G} for the aggregates produced in this example is from approximately 0.99 to 1.03, indicating that the aggregates were composed of carbon allotropes with a high degree of order.

FIG. 8D and FIG. 8E show example TEM images of the carbon nanoparticles after size reduction by grinding in a ball mill. The ball milling was performed in cycles with a 3 minute counter-clockwise grinding step, followed by a 6 minute idle step, followed by a 3 minute clockwise grinding step, followed by a 6 minute idle step. The grinding steps were performed using a rotation speed of 400 rpm. The milling media was zirconia and ranged in size from 0.1 mm to 10 mm. The total size reduction processing time was from 60 minutes to 120 minutes. After size reduction, the aggregate particles produced in this example had a particle size of approximately 1 μm to 5 μm. The carbon nanoparticles after size reduction are connected MWSFs with layers of graphene coating the connected MWSFs.

FIG. 8F shows a Raman spectrum from these aggregates after size reduction taken with a 532 nm incident light. The I_{D}/I_{G} for the aggregate particles in this example after size reduction is approximately 1.04. Additionally, the particles after size reduction had a Brunauer, Emmett and Teller (BET) specific surface area of approximately 40 m²/g to 50 m²/g.

The purity of the aggregates produced in this sample were measured using mass spectrometry and x-ray fluorescence (XRF) spectroscopy. The ratio of carbon to other elements, except for hydrogen, measured in 16 different batches was from 99.86% to 99.98%, with an average of 99.94% carbon.

In this example, carbon nanoparticles were generated using a thermal hot-wire processing system. The precursor material was methane, which was flowed from 1 slm to 5 slm. With these flow rates and the tool geometry, the resonance time of the gas in the reaction chamber was from approximately 20 second to 30 seconds, and the carbon particle production rate was from approximately 20 g/hr.

Further details pertaining to such a processing system can be found in the previously mentioned U.S. Pat. No. 9,862,602, titled “CRACKING OF A PROCESS GAS.”

FIG. 8G, FIG. 8H and FIG. 8I show TEM images of as-synthesized carbon nanoparticles of this example. The carbon nanoparticles contain connected multi-walled spherical fullerenes (MWSFs) with layers of graphene coating the connected MWSFs. The ratio of multi-walled fullerenes to graphene allotropes in this example is approximately 30% due to the relatively long resonance times allowing thicker, or more, layers of graphene to coat the MWSFs. No catalyst was used in this process, and therefore, there is no central seed containing contaminants. The as-synthesized aggregate particles produced in this example had particle sizes of approximately 10 μm to 500 μm. FIG. 8J shows a Raman spectrum from the aggregates of this example. The Raman signature of the as-synthesized particles in this example is indicative of the thicker graphene layers which coat the MWSFs in the as-synthesized material. Additionally, the as-synthesized particles had a Brunauer, Emmett and Teller (BET) specific surface area of approximately 90 m²/g to 100 m²/g.

FIG. 8K and FIG. 8L show TEM images of the carbon nanoparticles of this example. Specifically, the images depict the carbon nanoparticles after performance of size reduction by grinding in a ball mill. The size reduction process conditions were the same as those described as pertains to the foregoing FIG. 8G through FIG. 8J. After size reduction, the aggregate particles produced in this example had a particle size of approximately 1 μm to 5 μm. The TEM images show that the connected MWSFs that were buried in the graphene coating can be observed after size reduction. FIG. 8M shows a Raman spectrum from the aggregates of this example after size reduction taken with 532 nm incident light. The I_{D}/I_{G} for the aggregate particles in this example after size reduction is approximately 1, indicating that the connected MWSFs that were buried in the graphene coating as-synthesized had become detectable in Raman after size reduction, and were well ordered. The particles after size reduction had a Brunauer, Emmett and Teller (BET) specific surface area of approximately 90 m²/g to 100 m²/g.

FIG. 8N is a scanning electron microscope (SEM) image of carbon aggregates showing the graphite and graphene allotropes at a first magnification. FIG. 8O is a SEM image of carbon aggregates showing the graphite and graphene allotropes at a second magnification. The layered graphene is clearly shown within the distortion (wrinkles) of the carbon. The 3D structure of the carbon allotropes is also visible.

The particle size distribution of the carbon particles of FIG. 8N and FIG. 8O is shown in FIG. 8P. The mass basis cumulative particle size distribution 806 corresponds to the left y-axis in the graph (Q³(x) [%]). The histogram of the mass particle size distribution 808 corresponds to the right axis in the graph (dQ³(x) [%]). The median particle size is approximately 33 μm. The 10th percentile particle size is approximately 9 μm, and the 90th percentile particle size is approximately 103 μm. The mass density of the particles is approximately 10 g/L.

The particle size distribution of the carbon particles captured from a multiple-stage reactor is shown in FIG. 8Q. The mass basis cumulative particle size distribution 814 corresponds to the left y-axis in the graph (Q³(x) [%]). The histogram of the mass particle size distribution 816 corresponds to the right axis in the graph (dQ³(x) [%]). The median particle size captured is approximately 11 μm. The 10th percentile particle size is approximately 3.5 μm, and the 90th percentile particle size is approximately 21 μm. The graph in FIG. 8Q also shows the number basis cumulative particle size distribution 818 corresponding to the left y-axis in the graph (Q° (x) [%]). The median particle size by number basis is from approximately 0.1 μm to approximately 0.2 μm. The mass density of the particles collected is approximately 22 g/L.

Returning to the discussion of FIG. 8P, the graph also shows a second set of example results. Specifically, in this example, the particles were size-reduced by mechanical grinding, and then the size-reduced particles were processed using a cyclone separator. The mass basis cumulative particle size distribution 810 of the size-reduced carbon particles captured in this example corresponds to the left y-axis in the graph (Q³(x) [%]). The histogram of the mass basis particle size distribution 812 corresponds to the right axis in the graph (dQ³(x) [%]). The median particle size of the size-reduced carbon particles captured in this example is approximately 6 μm. The 10th percentile particle size is from 1 μm to 2 μm, and the 90th percentile particle size is from 10 μm to 20 μm.

Further details pertaining to making and using cyclone separators can be found in U.S. patent application Ser. No. 15/725,928, filed Oct. 5, 2017, titled “MICROWAVE REACTOR SYSTEM WITH GAS-SOLIDS SEPARATION”, which is hereby incorporated by reference in its entirety.

### High Purity Carbon Allotropes Produced Using Microwave Reactor Systems

In some cases, carbon particles and aggregates containing graphite, graphene and amorphous carbon can be generated using a microwave plasma reactor system using a precursor material that contains methane, or contains isopropyl alcohol (IPA), or contains ethanol, or contains a condensed hydrocarbon (e.g., hexane). In some other examples, the carbon-containing precursors are optionally mixed with a supply gas (e.g., argon). The particles produced in this example contained graphite, graphene, amorphous carbon and no seed particles. The particles in this example had a ratio of carbon to other elements (other than hydrogen) of approximately 99.5% or greater.

In one particular example, a hydrocarbon was the input material for the microwave plasma reactor, and the separated outputs of the reactor comprised hydrogen gas and carbon particles containing graphite, graphene and amorphous carbon. The carbon particles were separated from the hydrogen gas in a multi-stage gas-solid separation system. The solids loading of the separated outputs from the reactor was from 0.001 g/L to 2.5 g/L.

FIG. 8R, FIG. 8S, and FIG. 8T are TEM images of as-synthesized carbon nanoparticles. The images show examples of graphite, graphene and amorphous carbon allotropes. The layers of graphene and other carbon materials can be clearly seen in the images.

The particle size distribution of the carbon particles captured is shown in FIG. 8U. The mass basis cumulative particle size distribution 820 corresponds to the left y-axis in the graph (Q³(x) [%]). The histogram of the mass particle size distribution 822 corresponds to the right axis in the graph (dQ³(x) [%]). The median particle size captured in the cyclone separator in this example was approximately 14 μm. The 10th percentile particle size was approximately 5 μm, and the 90th percentile particle size was approximately 28 μm. The graph in FIG. 8U also shows the number basis cumulative particle size distribution 824 corresponding to the left y-axis in the graph (Q⁰(x) [%]). The median particle size by number basis in this example was from approximately 0.1 μm to approximately 0.2 μm.

FIG. 8V, FIG. 8W, and FIGS. 8X, and 8Y are images that show three-dimensional carbon-containing structures that are grown onto other three-dimensional structures. FIG. 8V is a 100× magnification of three-dimensional carbon structures grown onto carbon fibers, whereas FIG. 8W is a 200× magnification of three-dimensional carbon structures grown onto carbon fibers. FIG. 8X is a 1601× magnification of three-dimensional carbon structures grown onto carbon fibers. The three-dimensional carbon growth over the fiber surface is shown. FIG. 8Y is a 10000× magnification of three-dimensional carbon structures grown onto carbon fibers. The image depicts growth onto the basal plane as well as onto edge planes.

More specifically, FIGS. 8V-8Y show example SEM images of 3D carbon materials grown onto fibers using plasma energy from a microwave plasma reactor as well as thermal energy from a thermal reactor. FIG. 8V shows an SEM image of intersecting fibers 831 and 832 with 3D carbon material 830 grown on the surface of the fibers. FIG. 8W is a higher magnification image (the scale bar is 300 μm compared to 500 μm for FIG. 8V) showing 3D carbon growth 830 on the fiber 832. FIG. 8X is a further magnified view (scale bar is 40 μm) showing 3D carbon growth 830 on fiber surface 835, where the 3D nature of the carbon growth 830 can be clearly seen. FIG. 8Y shows a close-up view (scale bar is 500 nm) of the carbon alone, showing interconnection between basal planes 836 and edge planes 834 of numerous sub-particles of the 3D carbon material grown on the fiber. FIGS. 8V-8Y demonstrate the ability to grow 3D carbon on a 3D fiber structure according to some embodiments, such as 3D carbon growth grown on a 3D carbon fiber.

In some embodiments, 3D carbon growth on fibers can be achieved by introducing a plurality of fibers into the microwave plasma reactor and using plasma in the microwave reactor to etch the fibers. The etching creates nucleation sites such that when carbon particles and sub-particles are created by hydrocarbon disassociation in the reactor, growth of 3D carbon structures is initiated at these nucleation sites. The direct growth of the 3D carbon structures on the fibers, which themselves are three-dimensional in nature, provides a highly integrated, 3D structure with pores into which resin can permeate. This 3D reinforcement matrix (including the 3D carbon structures integrated with high aspect ratio reinforcing fibers) for a resin composite results in enhanced material properties, such as tensile strength and shear, compared to composites with conventional fibers that have smooth surfaces and which smooth surfaces typically delaminate from the resin matrix.

### Functionalizing Carbon

In some embodiments, carbon materials, such as 3D carbon materials described herein, can be functionalized to promote adhesion and/or add elements such as oxygen, nitrogen, carbon, silicon, or hardening agents. In some embodiments, the carbon materials can be functionalized in situ-that is, within the same reactor in which the carbon materials are produced. In some embodiments, the carbon materials can be functionalized in post-processing. For example, the surfaces of fullerenes or graphene can be functionalized with oxygen- or nitrogen-containing species which form bonds with polymers of the resin matrix, thus improving adhesion and providing strong binding to enhance the strength of composites.

Embodiments include functionalizing surface treatments for carbon (e.g., CNTs, CNO, graphene, 3D carbon materials such as 3D graphene) utilizing plasma reactors (e.g., microwave plasma reactors) described herein. Various embodiments can include in situ surface treatment during creation of carbon materials that can be combined with a binder or polymer in a composite material. Various embodiments can include surface treatment after creation of the carbon materials while the carbon materials are still within the reactor.

### Further Embodiments

In various embodiments, composite inks combining GNPs with 3DG may demonstrate synergistic improvements in conductivity. For example, a mixture of 95% GNP M-5 and 5% 3DG may achieve a conductivity of 1,257 S/m after drying at 120° C., significantly higher than either component alone (339 S/m and 303 S/m). Post-annealing at 450° C., this composite may reach a conductivity of 3,888 S/m.

Similar enhancements may be observed with other GNP types, including GNP M-25 and GNP H-25, where the addition of 5% 3DG can consistently boost conductivity compared to GNP-only and/or 3DG-only formulations. This synergistic effect may be broadly applicable across multiple GNP types, suggesting a general principle for enhancing the conductivity of carbon-based inks.

The processing method for these conductive inks may involve mixing the carbon materials with polyvinylpyrrolidone (PVP) as a binder and N-Methyl-2-pyrrolidone (NMP) as a solvent. The ratio of carbon to solvent can be optimized for each GNP type, potentially ranging from 1:10 to 1:15 by weight. It is acknowledged, however, that other methods and processes may be used to mix the carbon materials.

In various embodiments, effective dispersion may be achieved through a combination of bath sonication and probe-tip sonication. The latter technique may be particularly used for larger GNP particles like M-25 and H-25, which may require more intensive processing to achieve uniform dispersion.

Mechanical flexibility tests may be conducted on thin films of the conductive inks deposited on polyimide substrates. Despite the potential removal of the binder during annealing, the carbon films can exhibit excellent adhesion with no delamination issues. The overall mechanical flexibility may be excellent, potentially attributed to both the thin nature of the carbon film (approximately 10 μm) and the flexibility of the polyimide substrate (140 μm).

Conductivity may remain stable after simple bending tests, indicating good potential for flexible electronic applications. This combination of high conductivity and mechanical flexibility can make these inks suitable for various printed electronics applications where both properties are required.

In various embodiments, the relationship between carbon composition and conductivity may be clearly demonstrated. It has also been observed that the ratio of GNP to 3DG can be optimized and observed across different GNP types, suggesting a robust formulation principle.

The significant improvements in conductivity, coupled with good mechanical properties and processability, may position these inks as promising candidates for various printed electronics applications. These applications could potentially include flexible displays, wearable electronics, or printed circuit boards where high conductivity and flexibility are crucial.

Further optimization of the ink formulations may be possible by adjusting parameters such as the type and amount of binder, solvent selection, and processing conditions. This could potentially lead to even higher conductivity values or improved mechanical properties, expanding the range of potential applications for these carbon-based conductive inks.

In various embodiments, the conductive ink formulations may exhibit a synergetic effect on electrical conductivity when mixing two types of carbon materials. This effect can be observed across multiple sets of data, demonstrating consistent improvements in conductivity beyond what might be expected from a simple mixture of the individual components. The combination of different carbon types, such as 3D graphene and graphene nanoplatelets, may lead to enhanced electron pathways and improved overall conductivity of the resulting ink.

The selection of appropriate carbon materials, binders, and solvents can play a role in optimizing the electrical properties of the conductive inks. In some embodiments, the use of specific carbon materials, combined with carefully chosen binders and solvents, may result in an order of magnitude improvement in electrical conductivity compared to conventional formulations. This significant enhancement in conductivity can potentially expand the range of applications for these conductive inks in various electronic and energy storage devices.

Post-processing techniques may further contribute to the improvement of electrical conductivity in the dried ink films. For instance, calendering can potentially increase the density of the conductive network and reduce inter-particle resistance. In some embodiments, the application of such post-processing techniques may lead to substantial improvements in conductivity, potentially reaching an order of magnitude increase compared to untreated films.

The flexibility and durability of the conductive ink films may be demonstrated through various mechanical tests. In certain embodiments, Lyten carbon films deposited on polyimide substrates can exhibit high flexibility without significant degradation of their electrical properties. This combination of flexibility and conductivity may make these inks particularly suitable for applications in flexible electronics, where maintaining electrical performance under bending or folding conditions is crucial.

The durability of the conductive ink films may be assessed through repeated bending or folding tests. In some embodiments, the carbon films on polyimide substrates may maintain their structural integrity and electrical conductivity even after multiple bending cycles. This durability can be attributed to the strong adhesion between the carbon film and the substrate, as well as the intrinsic flexibility of the carbon network formed during the drying and post-processing stages.

In various embodiments, the synergetic effects of mixed carbon types, optimized formulations, and post-processing techniques may combine to produce conductive inks with exceptional electrical and mechanical properties. These inks can potentially offer a balance of high conductivity, flexibility, and durability that makes them suitable for a wide range of applications in printed and flexible electronics. The ability to tailor the ink properties through careful selection of materials and processing methods may allow for customization to meet specific application requirements.

In various embodiments, the conductive ink composition may comprise a combination of two different types of carbon materials. This combination can potentially yield synergistic effects, particularly in terms of electrical conductivity and mechanical properties. The first type of carbon may be configured to provide high conductivity, while the second type may be designed to enhance other properties such as mechanical durability or flexibility.

The ink composition may include a binder and a solvent in addition to the carbon materials. In some embodiments, the binder may be polyvinylpyrrolidone (PVP) and the solvent may be N-Methyl-2-pyrrolidone (NMP). The ratio of carbon to solvent may be optimized for different types of carbon materials, potentially ranging from 1:10 to 1:15 by weight.

In some embodiments, the ink may be applied to a substrate to form a conductive film. The substrate may be a flexible material such as polyimide. After application, the ink may undergo a drying process. This process can remove the solvent and initiate the formation of a conductive network within the film. In certain embodiments, a subsequent annealing process may be performed at higher temperatures. This annealing step may further enhance the conductivity of the film by improving the connections between carbon particles and potentially removing the binder.

The resulting conductive films may exhibit a combination of high electrical conductivity and good mechanical properties. In various embodiments, the conductivity of the films may be significantly higher than that achieved with either type of carbon material alone, demonstrating the synergistic effect of the combination. The mechanical properties may include excellent flexibility, with the films potentially maintaining their conductivity even after repeated bending or flexing. This combination of electrical and mechanical properties may make these films particularly suitable for applications in flexible electronics.

In some embodiments, additional post-processing techniques may be applied to further enhance the properties of the conductive films. These may include calendaring, which involves compressing the film between rollers. This process can potentially increase the density of the conductive network within the film, leading to improved electrical conductivity. Another potential post-processing technique may include an annealing process, which may remove the binder and further improve the connections between carbon particles.

The conductive ink compositions may be used to create sensors with unique properties. In various embodiments, the sensor may comprise multiple layers of the carbon film. The combination of different carbon types in specific proportions may allow for the creation of a film with distinct conductive “islands” connected by areas of lower conductivity. This structure may exhibit enhanced reflectivity compared to that of a uniform conductive film.

In some embodiments, the removal of the binder during the annealing process may affect the flexibility of the resulting film. While the exact effect may depend on the specific materials and processes used, the removal of the soft binder material could potentially lead to changes in the film's mechanical properties. These changes may need to be balanced against the potential improvements in electrical conductivity that can result from the annealing process.

The combination of different carbon types in the ink composition may allow for tailoring of various properties beyond just electrical conductivity. In some embodiments, the ink may be formulated to provide a balance of properties such as wear resistance, flexibility, and conductivity. This may allow for the creation of sensors or other devices that can maintain their electrical performance under challenging mechanical conditions, such as repeated bending or flexing.

In various embodiments, the sensor may comprise a plurality of layers of carbon film. Each layer of carbon film may include a mixture of a first carbon material comprising graphene nanoplatelets, a second carbon material comprising three-dimensional graphene, and a binder. The combination of these materials in specific proportions may produce a combined physical improvement compared to sensors containing only one of the carbon materials. This physical improvement may include, but is not limited to, synergistic improvements in electrical conductivity, enhanced flexibility, improved mechanical durability, and unique ensemble effects between the carbon materials.

The synergistic improvement in electrical conductivity may result in other results, allowing for fine-tuning of the sensor's electrical and mechanical properties. The specific ratio may be selected based on the intended application and desired sensor characteristics.

The sensor may be constructed on a substrate, which may comprise a flexible material. The plurality of layers of carbon film may be disposed on this substrate, allowing for the creation of flexible sensors. This flexibility, combined with the unique properties of the carbon film, may enable the sensor to detect various physical parameters. In some embodiments, the sensor may be configured to detect at least one of pressure, strain, or temperature, making it suitable for a wide range of applications in wearable electronics, structural health monitoring, or environmental sensing.

In various embodiments, the conductive ink composition may incorporate different types of graphene nanoplatelets (GNPs) and 3D graphene (3DG) to achieve desired electrical and mechanical properties. The GNPs may include varieties such as GNP M-5, which has a particle size of approximately 5 μm and a particle thickness of 6-8 nm; GNP M-25, featuring a larger particle size of about 25 μm while maintaining a thickness of 6-8 nm; and GNP H-25, which also has a particle size of around 25 μm but with an increased thickness of approximately 15 nm. These GNPs may be combined with 3DG, a unique form of graphene with a three-dimensional structure, in various ratios to create ink formulations with tailored characteristics. The specific combination of these materials may allow for fine-tuning of properties such as reflectivity, conductivity, flexibility, and/or durability, potentially enabling the creation of sensors or other devices with performance characteristics that surpass those achievable with any single type of graphene material.

### Improvement Over Prior Art Systems

In various embodiments, the conductive ink compositions described herein may offer significant improvements over prior art systems. While conventional graphene-based inks typically focus on maximizing conductivity through the use of a single, highly purified form of graphene, the present approach may utilize a combination of two distinct types of carbon materials. This combination can potentially yield synergistic effects that are not achievable with single-component systems. For instance, one type of carbon may be optimized for high conductivity, while the other may enhance mechanical properties such as flexibility or wear resistance. The resulting ink may exhibit a balance of properties that surpasses what can be achieved with either component alone.

The non-obvious nature of this improvement may be evident in the counterintuitive approach to enhancing overall performance. Conventional wisdom in the field often suggests that the highest conductivity would be achieved by using the purest, most conductive form of graphene possible. However, the present approach demonstrates that intentionally incorporating a second, potentially less conductive carbon material can lead to superior overall performance. This synergistic effect may not only enhance conductivity but also improve other critical properties such as reflectivity, flexibility, durability, and processability. Such a multi-faceted improvement may expand the potential applications for these conductive inks beyond what is possible with conventional single-component systems.

Furthermore, the described ink compositions may offer unique advantages in specific applications, such as sensors. In some embodiments, the combination of different carbon types may allow for the creation of a film structure with distinct conductive “islands” connected by regions of lower conductivity. This arrangement may enable the sensor to exhibit reflectivity at conductivity levels that would not typically support such behavior in a uniform conductive film. This capability represents a non-obvious improvement over prior art systems, potentially enabling new sensor designs and applications that were not previously feasible. The ability to fine-tune the balance between conductivity, flexibility, and other properties through the careful selection and combination of carbon materials may provide a level of control and versatility that surpasses conventional systems.

Still yet, in various embodiments, the improvement in conductive ink compositions may extend beyond simply combining two different types of carbon materials. The unique structural arrangement of these materials within the ink and resulting film can play a crucial role in achieving the desired properties. Specifically, the carbon materials may not need to be intimately bound or integrated at the molecular level. Instead, the ink composition may form a structure where islands of one type of carbon material are surrounded by a matrix of the other type. This arrangement can allow each material to maintain its distinct properties while still contributing to the overall performance of the ink.

This island-matrix structure may yield benefits that are not achievable through a more homogeneous mixture of carbon types. For example, highly conductive islands of one carbon type may provide excellent electrical pathways, while the surrounding matrix of a different carbon type may offer enhanced flexibility, wear resistance, or adhesion to the substrate. The interfaces between these distinct regions may create unique electrical and mechanical properties that are not present in either material alone. This structural arrangement may allow for the tailoring of ink properties to specific applications, such as sensors that require both high conductivity and mechanical durability. The ability to achieve these properties through structural arrangement rather than chemical bonding or intimate mixing may represent a non-obvious approach to improving conductive ink performance.

### Use-Case Scenarios

In one potential use case scenario, the conductive ink composition may be applied in the manufacturing of flexible wearable health monitors. The ink may be used to print sensor arrays and conductive traces directly onto a thin, flexible polyimide substrate that can conform to the contours of the human body. The unique combination of carbon materials in the ink may allow for the creation of highly conductive pathways for signal transmission, while also providing the durability to withstand repeated flexing and stretching as the wearer moves. The island-matrix structure of the carbon materials may enable the sensor to maintain its electrical properties even when subjected to significant bending or twisting, potentially allowing for more reliable and continuous health monitoring in real-world conditions. This application may take advantage of both the high conductivity and the mechanical resilience offered by the novel ink composition, potentially enabling more comfortable and longer-lasting wearable devices than those made with conventional conductive materials.

Another potential use case may involve the production of large-area, flexible gas sensors for environmental monitoring. In this scenario, the conductive ink may be used to create an array of interdigitated electrodes on a flexible substrate that can be wrapped around pipes or vessels in industrial settings. The specific combination of carbon materials in the ink may allow for the creation of a sensing layer that is both highly sensitive to target gas molecules and resistant to harsh environmental conditions. The island-matrix structure of the carbon materials may provide a high surface area for gas adsorption, potentially enhancing sensitivity, while also offering the durability to withstand exposure to corrosive atmospheres or mechanical stress. The flexibility of the resulting sensor may allow it to be easily installed in hard-to-reach areas or on curved surfaces, while its durability may enable long-term deployment with minimal maintenance. This application may leverage the unique balance of electrical, chemical, and mechanical properties offered by the novel ink composition to create sensors that are more versatile and robust than those made with traditional conductive materials.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

The embodiments described herein included the one or more modes known to the inventor for carrying out the claimed subject matter. Of course, variations of those embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

What is claimed is:

1. A sensor, comprising:
a plurality of layers of graphene ink configured such that each layer of graphene ink comprises a mixture of:
large, conductive patches of graphene and,
low conductivity interstitial carbon material,
wherein the low conductivity interstitial carbon material bridges the large, conductive patches of graphene.

2. The sensor of claim 1, wherein the graphene ink comprises a first type of graphene configured for conductivity and a second type of graphene configured for wear and tear resistance.

3. The sensor of claim 2, wherein the first type of graphene and the second type of graphene are mixed together prior to application on the sensor.

4. The sensor of claim 2, wherein the first type of graphene and the second type of graphene are applied in separate layers on the sensor.

5. The sensor of claim 4, wherein the first type of graphene is applied before the second type of graphene.

6. The sensor of claim 4, wherein the second type of graphene is applied before the first type of graphene.

7. The sensor of claim 1, wherein the low conductivity interstitial carbon material comprises a carbon-based filler material.

8. The sensor of claim 1, wherein the large, conductive patches of graphene and the low conductivity interstitial carbon material are arranged in a non-overlapping pattern.

9. The sensor of claim 1, wherein the sensor is at least one of a resonant sensor, a vapor sensor, a gas sensor, a biosensor, or a printed label sensor.

10. The sensor of claim 1, wherein the sensor is configured to detect changes in at least one of: environmental conditions, physical conditions, chemical conditions, biological conditions, electrical conditions, thermal conditions, mechanical conditions, or optical conditions.

11. A sensor, comprising:
a graphene ink formulation, wherein the graphene ink formulation comprises:
a first type of graphene configured for conductivity and
a second type of graphene configured for wear and tear resistance,
wherein the first type of graphene and the second type of graphene are mixed together prior to application on the sensor.

12. The sensor of claim 11, wherein the first type of graphene is configured for high conductivity and the second type of graphene is configured for high elasticity.

13. The sensor of claim 11, wherein the first type of graphene and the second type of graphene are mixed together prior to application on the sensor.

14. The sensor of claim 11, wherein the first type of graphene and the second type of graphene are applied in separate layers on the sensor.

15. A sensor, comprising:
a film comprising a graphene ink formulation, wherein the graphene ink formulation comprises:
a first type of graphene configured for conductivity and
a second type of graphene configured for wear and tear resistance,
wherein the first type of graphene and the second type of graphene are applied in separate layers on the sensor.

16. The sensor of claim 15, wherein the first type of graphene is configured for high conductivity and the second type of graphene is configured for high elasticity.

17. The sensor of claim 15, wherein the first type of graphene and the second type of graphene are mixed together prior to application on the sensor.

18. The sensor of claim 15, wherein each of the first type of graphene and the second type of graphene are mixtures each containing two or more types of graphene.

19. A sensor, comprising:
a plurality of layers of carbon film configured such that each layer of carbon film comprises a mixture of:
a first carbon material comprising graphene nanoplatelets;
a second carbon material comprising three-dimensional graphene; and
a binder;
wherein the first carbon material and the second carbon material are combined in proportions that produce a combined physical improvement compared to sensors containing only the first carbon material or only the second carbon material.

20. The sensor of claim 19, wherein the combined physical improvement includes a synergistic improvement in electrical conductivity.

21. The sensor of claim 20, wherein the synergistic improvement includes a reflective property that is based on the interaction between the first carbon material and the second carbon material, which is not achievable with either carbon material alone.

22. The sensor of claim 19, wherein the combined physical improvement includes enhanced flexibility.

23. The sensor of claim 19, wherein the combined physical improvement includes an ensemble effect between the first carbon material and the second carbon material.

24. The sensor of claim 23, wherein the ensemble effect comprises a network of interconnected conductive pathways formed by the three-dimensional graphene structures bridging gaps between the graphene nanoplatelets, wherein the network increases electron transport and overall conductivity of the carbon film.

25. The sensor of claim 19, wherein the combined physical improvement includes improved mechanical durability.

26. The sensor of claim 19, wherein the first carbon material comprises aggregates of graphene nanoplatelets with an aggregate size of less than 25 μm and a thickness of less than 15 nm.

27. The sensor of claim 19, wherein the second carbon material comprises three-dimensional graphene with a specific surface area greater than 250 m2/g.

28. The sensor of claim 19, wherein the ratio of the first carbon material to the second carbon material is between 70:30 and 99:1 by weight.

29. The sensor of claim 19, further comprising a substrate, wherein the plurality of layers of carbon film are disposed on the substrate, and wherein the substrate comprises a flexible material.

30. The sensor of claim 19, wherein the sensor is configured to detect at least one of pressure, strain, or temperature.
